SEQUENCE LISTING (40 sequences)

SEQ ID NO: 1 - DNA, 2187 bp, Homo sapiens
atggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatga

SEQ ID NO: 2 - DNA, 2112 bp, Artificial Sequence
  Synthetic construct
atgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtga

SEQ ID NO: 3 - DNA, 2187 bp, Artificial Sequence
  Synthetic construct
atggccgggttttgggtgggcacggccccgctcgtagcagctggcaggcgggggcgatggcccccccagcagcttatgcttagtgccgccttgcggacgctgaagcacgtcctttactactctagacagtgccttatggtaagccgaaatttgggaagtgtaggttatgatcccaacgagaagacctttgataagatactggttgctaaccgaggggagatagcgtgtcgagttattcgcacctgtaagaagatgggaattaaaaccgtggccatccatagcgatgtcgacgcttccagtgtgcacgttaaaatggccgacgaggccgtatgcgtggggcctgcccctacctctaagtcatacctgaacatggatgcaattatggaagctattaagaagactcgggcgcaggctgtccaccctggatatggatttctttctgagaataaggagtttgcccggtgtctggcggcagaagacgtcgtattcattggaccggatacgcacgctatccaagccatgggagataagatcgagagcaagctcctggctaagaaagctgaagtgaacaccattcctggctttgacggcgtggtgaaggacgcagaggaagctgttcgcatcgcccgcgaaattggatatcccgtgatgataaaagcatctgcgggggggggcgggaagggcatgagaattgcctgggatgatgaagaaactagagatggtttccgcttgtcttctcaggaagccgcatcatcctttggagatgaccgattgctcatagagaaatttatcgacaatccacggcatattgagatccaagtgcttggcgacaagcacggtaacgcgctttggctcaacgaacgagagtgttcaatccagaggaggaaccagaaggttgtagaagaagcaccatctattttcctcgacgcagaaactcggcgggctatgggggaacaagcagtggcactggctcgagccgttaaatattctagtgcggggacagtagaattcctcgtagatagcaagaagaatttttattttcttgagatgaatacgcgccttcaagtggaacacccagtcacggaatgtataactggccttgacttggttcaggagatgatacgggtggctaagggttatcctcttcggcataagcaggctgatattcgcataaatgggtgggcggtcgagtgcagagtttatgctgaggacccatacaagtcattcggacttccttctataggcagactgtcacaatatcaagagccacttcatctcccaggtgtaagagtagattccggaatacaacctggctccgatatatctatttactatgatccaatgattagtaagttgattacatatgggagtgatcggaccgaagctttgaagcggatggcggacgcgctggataactacgtgataaggggtgtcacgcacaatatagctctgctgagggaggtaattatcaacagtcggttcgtgaagggtgacattagcactaagttcctctccgacgtgtacccagacggttttaaagggcacatgcttactaagtccgaaaagaatcaactgttggctattgcgtcttccctttttgttgctttccaactgcgcgcgcagcatttccaggagaatagcagaatgcccgttatcaaacctgatattgcgaactgggaattgtcagttaagctgcatgataaggtgcataccgtagtggctagtaataacggaagcgttttttccgttgaagtagacggctccaagcttaatgtgacgagcacatggaaccttgcctctccactgcttagtgtgagtgtggacggaacgcagaggacagttcaatgcctgagtcgggaagcgggaggtaacatgagtatacaattcctcggaaccgtctataaagttaacattttgacgagattggcggctgaactgaataagttcatgctcgagaaagtgactgaggacacttcaagcgtactgaggagccctatgccgggggttgtcgtagcagtgtctgttaagccaggagatgcggtggcagaaggccaagaaatttgcgtgattgaggcaatgaaaatgcagaactcaatgaccgccggaaaaacgggcacggtcaaatctgtgcattgtcaggcaggcgacacagtcggcgagggtgatctcctggtagagttggaatga

SEQ ID NO: 4 - DNA, 2187 bp, Artificial Sequence
  Synthetic construct
atggccggcttctgggtggggactgctccccttgtcgccgcaggacgcagaggccgctggcctcctcagcagctcatgctctcagcagctctgaggaccctgaaacacgtgctttactacagtcgacagtgtctgatggtgtctaggaacctgggtagcgtgggctatgatcccaatgaaaagacctttgacaaaatactggtcgctaatagaggggaaattgcttgtcgcgtgatacggacgtgcaagaagatgggtatcaaaaccgtggcaattcactctgacgttgatgcttcctcagtgcatgtaaagatggcggatgaggctgtttgcgtgggtccagcacctacaagcaagagctatctcaacatggatgccatcatggaagctatcaagaaaacccgtgcacaagctgtgcatccagggtatggctttctctccgagaacaaagaatttgcccggtgtctggcagcggaggacgtggtgttcattgggcctgatacgcatgcaattcaagccatgggcgataagattgagagcaagctgcttgctaagaaagcagaagttaacacaatcccaggctttgacggcgttgtcaaagacgccgaagaagcggtacgtattgcccgagaaatcggctaccccgttatgatcaaggcgtcagccggaggtggaggaaaagggatgaggattgcctgggatgacgaggagactagggatgggttccggctctccagtcaggaagcagcatcttcttttggtgacgatagactgctgatagagaaattcatcgacaaccctcgacacattgaaatccaggtactgggagacaaacacggaaatgcactttggctcaatgaacgcgagtgctccattcagcgcaggaaccagaaagtggtcgaggaagcaccctccatcttcctggatgccgagacaaggcgcgctatgggcgagcaggccgttgcactcgctagagccgtgaagtactcttctgcgggtaccgtggaatttctggtagacagcaagaagaacttctatttcctggagatgaatacccggctgcaagtcgagcatccagtcactgagtgtataactggcctggacctggtacaggaaatgattcgtgtagcgaagggatacccgctccggcacaaacaagccgacattcgcatcaatgggtgggctgtggagtgcagagtctatgcagaggatccctataagtccttcgggcttccctccataggcaggcttagtcagtatcaggagccattgcacttgcctggcgtcagggtggactccggcatccaaccgggcagcgacatttcaatttactacgatcccatgatcagcaagttgattacctatggatctgaccggacagaggctctgaagagaatggccgacgccctggacaattacgtgataagaggagtgacacacaacattgccctgttgcgggaggtgatcatcaatagcagattcgtgaagggtgacatctccaccaagttcctgagtgacgtataccccgacggctttaaggggcatatgctgacaaagtcagagaagaatcaactcctcgcaatagccagtagcctgtttgttgccttccagctgagggctcagcacttccaggagaatagcagaatgcccgttatcaaacctgatatcgcgaattgggaattgagcgtgaagctgcacgataaagttcatactgttgtggcctcaaacaatggaagcgtctttagcgtggaggtcgatggatccaaactgaacgtgaccagtacctggaatttggccagtccgctgttgtctgtctccgtggatggaacgcaacgaactgtgcagtgtctgtctcgcgaagccggaggcaacatgagcattcagtttctcgggactgtgtacaaagtcaacatcctgacccgactggctgccgagctgaacaaatttatgcttgagaaagtcactgaggatacgtctagcgtccttcggagtcctatgccaggggtggtggtggccgtttcagtcaaaccaggtgatgccgtagccgaaggtcaggaaatctgcgttatcgaggctatgaagatgcagaacagcatgacagccgggaaaaccggaacagtgaagtcagttcattgccaggctggggacacagtcggcgagggcgatttgctggtggaactggaatga

SEQ ID NO: 5 - DNA, 2187 bp, Artificial Sequence
  Synthetic construct
atggccggattttgggttggaacagctcctctggtggccgctgggagaagaggaagatggcctcctcagcagctgatgctgtctgccgctctgagaaccctgaaacacgtgctgtactacagccggcagtgcctgatggtgtccagaaatctgggcagcgtgggctacgaccccaacgagaaaaccttcgacaagatcctggtggccaaccggggagagatcgcctgcagagtgatccggacctgcaagaagatgggcatcaagaccgtggccatccactccgatgtggatgcctctagcgtgcacgtgaaaatggccgatgaggccgtgtgtgtgggccctgctcctacaagcaagagctacctgaacatggacgccatcatggaagccattaagaaaacaagagcccaggccgtgcatcccggctacggatttctgagcgagaacaaagaatttgcccggtgcctggccgccgaggacgtggtgtttattggccctgatacacacgccatccaggccatgggcgataagatcgagtctaagctgctggccaagaaagccgaagtgaacacaatccccggcttcgacggcgtggtcaaggatgctgaagaagccgtgcggatcgccagagaaatcggctaccccgtgatgatcaaagcctctgctggcggaggcggcaagggaatgagaatcgcctgggacgacgaagagacacgcgacggctttagactgagcagccaagaagccgccagctccttcggagatgacagactgctgatcgagaagttcatcgacaaccccagacacatcgagatccaggtgctgggcgacaagcacggaaatgccctgtggctgaacgagagagagtgcagcatccagcggcggaaccagaaagtggtggaagaggcccctagcatcttcctggacgccgaaactcggagagccatgggagaacaggctgtggctctggctagagccgtgaagtatagcagcgccggcaccgtggaatttctggtggacagcaagaagaacttctacttcctcgagatgaacacccggctgcaggtcgagcaccctgtgaccgagtgtatcacaggcctggacctggtgcaagagatgatcagagtggccaagggctaccctctgagacacaagcaggccgacatccggatcaatggctgggccgttgagtgcagagtgtacgccgaggatccctacaagagcttcggcctgcctagcatcggccggctgtctcagtatcaagagccactgcatctgcccggcgtcagagtggattctggaatccagcctggcagcgacatcagcatctactacgaccctatgatctccaagctgatcacctacggcagcgaccggacagaggccctgaagagaatggctgacgccctggacaactacgtgatcagaggcgtgacccacaatatcgccctgctgcgggaagtgatcatcaacagcagattcgtgaaaggcgatatcagcaccaagtttctgtccgacgtgtaccccgacggcttcaagggacacatgctgaccaagagcgagaagaaccagctgctcgccattgcctccagcctgtttgtggcctttcagctgagagcccagcacttccaagagaacagcagaatgcccgtgatcaagcccgatatcgccaactgggagctgagcgtgaagctgcacgataaggtgcacacagtggtggccagcaacaacggctccgtgttcagcgtggaagtggacggcagcaagctgaacgtgacctccacctggaatctggcctctccactgctgtccgtgtctgtggatggcacccagagaaccgtgcagtgtctgagcagagaagcaggcggcaatatgagcatccagtttctgggcaccgtgtacaaagtgaacatcctgaccagactggccgctgagctgaacaagttcatgctggaaaaagtgaccgaggacaccagcagcgtgctgagatctcctatgcctggtgtcgtggtggccgtgtcagtgaaacctggggatgctgtggccgagggccaagagatctgtgtgatcgaggccatgaagatgcagaacagcatgaccgccggcaagaccggcacagtgaagtctgtgcattgtcaggccggcgatacagtcggagaaggcgatctgctggtggaactggaatga

SEQ ID NO: 6 - DNA, 2187 bp, Artificial Sequence
  Synthetic construct
atggccggcttctgggtgggcaccgcccccctggtggccgccggcagaagaggcagatggcccccccagcagctgatgctgagcgccgccctgagaaccctgaagcacgtgctgtactacagcagacagtgcctgatggtgagcagaaacctgggcagcgtgggctacgaccccaacgagaagaccttcgacaagatcctggtggccaacagaggcgagatcgcctgcagagtgatcagaacctgcaagaagatgggcatcaagaccgtggccatccacagcgacgtggacgccagcagcgtgcacgtgaagatggccgacgaggccgtgtgcgtgggccccgcccccaccagcaagagctacctgaacatggacgccatcatggaggccatcaagaagaccagagcccaggccgtgcaccccggctacggcttcctgagcgagaacaaggagttcgccagatgcctggccgccgaggacgtggtgttcatcggccccgacacccacgccatccaggccatgggcgacaagatcgagagcaagctgctggccaagaaggccgaggtgaacaccatccccggcttcgacggcgtggtgaaggacgccgaggaggccgtgagaatcgccagagagatcggctaccccgtgatgatcaaggccagcgccggcggcggcggcaagggcatgagaatcgcctgggacgacgaggagaccagagacggcttcagactgagcagccaggaggccgccagcagcttcggcgacgacagactgctgatcgagaagttcatcgacaaccccagacacatcgagatccaggtgctgggcgacaagcacggcaacgccctgtggctgaacgagagagagtgcagcatccagagaagaaaccagaaggtggtggaggaggcccccagcatcttcctggacgccgagaccagaagagccatgggcgagcaggccgtggccctggccagagccgtgaagtacagcagcgccggcaccgtggagttcctggtggacagcaagaagaacttctacttcctggagatgaacaccagactgcaggtggagcaccccgtgaccgagtgcatcaccggcctggacctggtgcaggagatgatcagagtggccaagggctaccccctgagacacaagcaggccgacatcagaatcaacggctgggccgtggagtgcagagtgtacgccgaggacccctacaagagcttcggcctgcccagcatcggcagactgagccagtaccaggagcccctgcacctgcccggcgtgagagtggacagcggcatccagcccggcagcgacatcagcatctactacgaccccatgatcagcaagctgatcacctacggcagcgacagaaccgaggccctgaagagaatggccgacgccctggacaactacgtgatcagaggcgtgacccacaacatcgccctgctgagagaggtgatcatcaacagcagattcgtgaagggcgacatcagcaccaagttcctgagcgacgtgtaccccgacggcttcaagggccacatgctgaccaagagcgagaagaaccagctgctggccatcgccagcagcctgttcgtggccttccagctgagagcccagcacttccaggagaacagcagaatgcccgtgatcaagcccgacatcgccaactgggagctgagcgtgaagctgcacgacaaggtgcacaccgtggtggccagcaacaacggcagcgtgttcagcgtggaggtggacggcagcaagctgaacgtgaccagcacctggaacctggccagccccctgctgagcgtgagcgtggacggcacccagagaaccgtgcagtgcctgagcagagaggccggcggcaacatgagcatccagttcctgggcaccgtgtacaaggtgaacatcctgaccagactggccgccgagctgaacaagttcatgctggagaaggtgaccgaggacaccagcagcgtgctgagaagccccatgcccggcgtggtggtggccgtgagcgtgaagcccggcgacgccgtggccgagggccaggagatctgcgtgatcgaggccatgaagatgcagaacagcatgaccgccggcaagaccggcaccgtgaagagcgtgcactgccaggccggcgacaccgtgggcgagggcgacctgctggtggagctggagtga

SEQ ID NO: 7 - DNA, 2187 bp, Artificial Sequence
  Synthetic construct
atggccggattttgggtcggaactgcaccacttgtcgctgccggtagaagaggaagatggccaccgcagcaactgatgttgagcgctgcactgcgcacactgaagcatgtgctgtactactcgcgccagtgtttgatggtgtccaggaatctcggctccgtgggctacgaccccaacgaaaagacttttgacaagatcctcgtggccaacagaggggaaattgcgtgccgcgtgattcggacttgcaagaagatgggaatcaagaccgtggccatacactccgatgtggacgcctcctccgtccacgtcaagatggctgatgaagccgtctgcgtgggaccggcgcctacttccaagtcgtaccttaacatggacgccatcatggaggccatcaagaaaaccagggcgcaggcggtgcatcctggctacggcttcctgtccgaaaacaaggagttcgcacggtgcctggccgccgaggacgtggtctttatcgggcccgacacccatgcaatccaggccatgggcgacaagatcgagtcgaagctgctggcgaagaaggcagaagtgaacactattcccgggttcgacggagtggtcaaagacgcggaagaggccgtccgaatcgcccgggagattggataccctgtgatgattaaggcctcggctggcggaggcggaaagggaatgcgcattgcctgggatgacgaagaaacccgggatggattccggctgagctcccaagaagccgcatcgtccttcggggacgatagactgctgatcgaaaagttcatcgacaacccaaggcacatcgaaatccaggtcctcggggacaagcatggaaacgccctgtggttgaacgagagagagtgctccattcaacggcgcaaccagaaggtcgtggaggaagccccctcgattttcctcgatgctgaaactcgccgggccatgggggagcaagcggtggccctggcccgcgcagtgaagtactcctcggccgggaccgtggagttcctggtggacagcaaaaagaacttctactttctcgagatgaacaccaggctccaagtggagcaccctgtgaccgaatgcatcactggacttgacctggtgcaggaaatgatccgcgtggccaagggataccccctgaggcacaagcaggccgacatcagaatcaacggttgggccgtggaatgtcgggtgtacgctgaggatccgtataagtccttcggcttgccgagcatcggacggctgtcacagtaccaggaacccctgcaccttcctggagtcagagtggactccggaatccaacctggttcggacatttccatctactacgatccgatgatctccaaactcattacctacggtagcgaccggaccgaggctctgaaacgcatggctgacgccctggacaactatgtcatccggggagtcactcacaatatcgcgctgctgcgcgaagtcatcattaatagccgcttcgtgaagggcgacatttccaccaagttcctgagcgacgtgtaccctgatggtttcaagggtcacatgctgactaagtccgagaagaaccagctcctcgctatcgcgtcctccctgtttgtggcgttccagctgagggcccagcacttccaagaaaactcaagaatgccggtcatcaagcccgacattgccaattgggaactgagcgtgaagctgcacgacaaagtgcacaccgtggtggccagcaacaacggctccgtgttctccgtggaagtggatgggtcaaagctgaacgtgaccagcacctggaacctggcgtccccgctcctgtcagtgtccgtggacggcactcagcggactgtgcagtgtttgtcccgggaagccgggggcaatatgagcatccagttcctcgggacggtgtacaaggtcaacatcctcactcggttggccgctgaactcaacaagttcatgctggaaaaggtcaccgaggacacctcctctgtgctgcggtcgcccatgccgggagtggtcgtggccgtgtccgtgaagcctggcgatgccgtggccgaaggtcaagaaatttgcgtgatcgaggccatgaagatgcagaactcgatgacggccggaaagaccggcaccgtcaaaagcgtgcactgccaggccggcgataccgtgggagagggcgatctgctcgtggaactcgaatga

SEQ ID NO: 8 - protein, 728 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 9 - DNA, 7362 bp, Artificial Sequence
  Synthetic construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggcggggttctgggtcgggacagcaccgctggtcgctgccggacggcgtgggcggtggccgccgcagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 10 - DNA, 7255 bp, Artificial Sequence
  Synthetic construct
ccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcgccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatcgaattccctgcaggtagagcatggctacgtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 11 - DNA, 6533 bp, Artificial Sequence
  Synthetic construct
gggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaatacgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 12 - DNA, 6530 bp, Artificial Sequence
  Synthetic construct
gggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaatacgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttattgcctttatatgcatgtatacaagcaaaacaggcttttactttctcgccaacttacaaggcctttctcagtaaacagtatagaccctttaccccgttgctcggcaacggcctggtctgtgccaagtgtttgctgacgcaacccccactggttggggcttggccataggccatcagcgcagcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccaggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 13 - DNA, 189 bp, Artificial Sequence
  Synthetic construct
cctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagag

SEQ ID NO: 14 - DNA, 318 bp, Artificial Sequence
  Synthetic construct
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgaga

SEQ ID NO: 15 - DNA, 251 bp, Artificial Sequence
  Synthetic construct
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 16 - DNA, 392 bp, Artificial Sequence
  Synthetic construct
tgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 17 - DNA, 133 bp, Artificial Sequence
  Synthetic construct
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 18 - DNA, 447 bp, Artificial Sequence
  Synthetic construct
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 19 - DNA, 165 bp, Artificial Sequence
  Synthetic construct
gtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcag

SEQ ID NO: 20 - DNA, 189 bp, Artificial Sequence; identical to SEQ ID NO: 13
  Synthetic construct

SEQ ID NO: 21 - DNA, 710 bp, Artificial Sequence
  Synthetic construct
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 22 - DNA, 1157 bp, Artificial Sequence
  Synthetic construct
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 23 - DNA, 100 bp, Artificial Sequence
  Synthetic construct
aggttaatttttaaaaagcagtcaaaagtccaagtggcccttggcagcatttactctctctgtttgctctggttaataatctcaggagcacaaacattcc

SEQ ID NO: 24 - DNA, 206 bp, Artificial Sequence
  Synthetic construct
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattcc

SEQ ID NO: 25 - DNA, 460 bp, Artificial Sequence
  Synthetic construct
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 26 - DNA, 666 bp, Artificial Sequence
  Synthetic construct
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccgggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 27 - DNA, 588 bp, Artificial Sequence
  Synthetic construct
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 28 - DNA, 63 bp, Artificial Sequence
  Synthetic construct
gggagtatattagtgctaatttccctccgtttgtcctagcttttctcttctgtcaaccccaca

SEQ ID NO: 29 - DNA, 81 bp, Artificial Sequence
  Synthetic construct
gggattcatgaaaatccactactccagacagacggctttggaatccaccagctacatccagctccctgagcagagttgaga

SEQ ID NO: 30 - DNA, 264 bp, Artificial Sequence
  Synthetic construct
gggacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaca

SEQ ID NO: 31 - DNA, 598 bp, Artificial Sequence
  Synthetic construct
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 32 - DNA, 726 bp, Artificial Sequence
  Synthetic construct
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 33 - DNA, 313 bp, Artificial Sequence
  Synthetic construct
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 34 - DNA, 235 bp, Artificial Sequence
  Synthetic construct
tttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 35 - DNA, 6607 bp, Artificial Sequence
  Synthetic construct
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 36 - DNA, 6047 bp, Artificial Sequence
  Synthetic construct
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgtcgagaattcagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 37 - DNA, 4657 bp, Artificial Sequence
  Synthetic construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagctagtaacggccgccagtgtgctggaattcggcttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggccctgcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgacatcacatttaaaagcatctcaggtaactatattttgaatttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttctgtacacgcgatccatcacactggcggccgctcgactggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 4791 bp, Artificial Sequence
  Synthetic construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtaccaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacagccaccatggcagggttctgggtcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggattccaggggtgtgtttcgtcgagatgcacgtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccagacaagagtgaggttgctcatcggtttaaagatttgggagaagaaaatttcaaagccttgtaagttaaaatattgatgaatcaaatttaatgtttctaatagtgttgtttattattctaaagtgcttatatttccttgtcatcagggttcagattctaaaacagtgctgcctcgtagagttttctgcgttgaggaagatattctgtatctgggctatccaataaggtagtcactggtcacatggctattgagtacttcaaatatgacaagtgcaactgagaaacaaaaacagcaattcgttgatcgaattccctgcaggtagagcatggctacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 9008 bp, Artificial Sequence
  Synthetic construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagtttgtacaaaaaagcaggctgccaccatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 40 - DNA, 9477 bp, Artificial Sequence
  Synthetic construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

(19) United States
(12) Patent Application Publication
(10) Pub. No.: US 2022/0251536 A1
(43) Pub. Date: Aug. 11, 2022

(54) SYNTHETIC GENES FOR THE TREATMENT OF PROPIONIC ACIDEMIA CAUSED BY MUTATIONS IN PROPIONYL-COA CARBOXYLASE ALPHA

(71) Applicant: The United States of America,as represented by the Secretary,Department of Health and Human Services, Bethesda, MD (US)

(72) Inventors: Charles P. Venditti, Bethesda, MD (US); Randy J. Chandler, Bethesda, MD (US)

(73) Assignee: The United States of America,as represented by the Secretary,Department of Health and Human Services, Bethesda, MD (US)

(21) Appl. No.: 17/620,331

(22) PCT Filed: Jun. 26, 2020
(86) PCT No.: PCT/US2020/039901

§ 371 Date: Dec. 17, 2021

Related U.S. Application Data
(60) Provisional application No. 62/867,374, filed on Jun. 27, 2019.

(51) Int. Cl. C12N 9/00; C12N 15/86; A61K 38/53; A61K 48/00; A61P 3/00

(52) U.S. Cl. CPC C12N 9/93; C12Y 604/01003; C12N 15/86; C12N 2830/008; A61K 48/005; A61P 3/00; C12N 2800/22; A61K 38/53

(57) ABSTRACT

Synthetic polynucleotides encoding human propionyl-CoA carboxylase alpha (synPCCA) and exhibiting augmented expression in cell culture and/or in a subject are described herein. Adeno-associated viral (AAV) gene therapy vectors encoding synPCCA successfully rescued the neonatal lethal phenotype displayed by propionyl-CoA carboxylase alpha (Pcca^{-/-}) deficient mice, lowered circulating methylcitrate levels in the treated animals, and resulted in prolonged hepatic expression of the product of the synPCCA transgene in vivo.

### PRIORITY DATA

This application claims the benefit of U.S. Provisional Application No. 62/867,374, filed Jun. 27, 2019, the entire disclosure of which is hereby incorporated by reference.

### STATEMENT OF GOVERNMENT INTEREST

The instant application was made with government support; the government has certain rights in this invention.

### SEQUENCE LISTING DATA

The Sequence Listing text document filed herewith, created Jun. 26, 2020, size 128 kilobytes, and named “NHGRI-12-PCT_ST25” is incorporated herein by reference in its entirety.

### FIELD OF THE INVENTION

The subject invention relates to engineering of the human propionyl-CoA carboxylase alpha gene (PCCA) so as to enhance expression and detection in eukaryotic cells. Compared to the wild-type human PCCA gene, the subject synthetic gene sequences (synPCCA) are codon-optimized to enhance expression upon administration and allow detection over the wild-type human PCCA gene by virtue of unique nucleic acid sequences composition.

### BACKGROUND

Propionic acidemia (PA) is an autosomal recessive metabolic disorder caused by mutations in either of PCCA or PCCB genes. The products of these genes form the alpha and beta subunits of the enzyme propionyl-CoA carboxylase (PCC), a critically important mitochondrial enzyme involved in the catabolism of branched chain amino acids. Specifically, propionyl-CoA carboxylase catalyzes the carboxylation of propionyl-CoA to D-methylmalonyl-CoA.

The results from an ongoing PA natural history study have revealed that in a large and diverse cohort of patients, approximately 50% have PA caused by PCCA mutations. Many PA patients present within the first few days to weeks of life with symptoms, and lethality can ensue if clinical recognition and treatment is delayed. Laboratory investigations show characteristic elevations of propionylcarnitine, 3-hydroxypropionate, and 2-methylcitrate (2-MC or MC). Milder patients can escape from early presentations but remain at risk for metabolic decompensation and late complications, especially cardiomyopathy. All individuals with PA can experience high mortality and disease related morbidity despite nutritional therapy. The failure of conventional medical and dietary management to treat PA has led to the use of elective liver transplantation as an alternative approach to stabilize metabolism and mitigate the risk of lethal metabolic decompensations.

### SUMMARY

The only treatments for PA currently available are dietary restrictions and elective liver transplantation. Patients still become metabolically unstable while on diet restriction and experience disease progression, despite medical therapy. These episodes result in numerous hospitalizations and can be fatal. The disclosure teaches a series of synthetic human propionyl-CoA carboxylase alpha (synPCCA) transgenes that can be used as a drug, via viral- or non-viral mediated gene delivery, to restore PCC function in PA patients, prevent metabolic instability, and ameliorate disease progression. Because this enzyme is important in other human disorders of branched chain amino acid oxidation, gene delivery of a synthetic PCCA gene might used to treat conditions other than PA.

Additionally, a synPCCA transgene can be used for the in vitro production of PA for use in enzyme replacement therapy for PA. Enzyme replacement therapy is accomplished by administration of the synthetic PCC protein, sub-cutaneously, intra-muscularly, intravenously, or by other therapeutic delivery routes.

Thus, in one aspect, the invention is directed to a synthetic propionyl-CoA carboxylase alpha gene (synPCCA) selected from the group consisting of:

a) a polynucleotide comprising the nucleic acid sequence of any one of SEQ ID NOs:2-7;
b) a polynucleotide having the nucleic acid sequence of any one of SEQ ID NOs:2-7;
c) a polynucleotide having a nucleic acid sequence with at least about 80% identity to the nucleic acid sequence of any one of SEQ ID NOs:2-7;
d) a polynucleotide encoding a polypeptide having the amino acid sequence of SEQ ID NO:8 or an amino acid sequence substantially identical to the amino acid sequence of SEQ ID NO:8, wherein the polynucleotide does not have the nucleic acid sequence of SEQ ID NO:1; and
e) a polynucleotide encoding an active fragment of the propionyl-CoA carboxylase (PCC) protein, wherein the polynucleotide in its entirety does not share 100% identity with a portion of the nucleic acid sequence of SEQ ID NO:1.
In one embodiment, the disclosure teaches a synthetic propionyl-CoA carboxylase subunit a (PCCA) polynucleotide (synPCCA) selected from the group consisting of: a polynucleotide comprising the nucleic acid sequence of any one of SEQ ID NOs: 2-7; a polynucleotide comprising a polynucleotide having a nucleic acid sequence with at least about 80% identity to the nucleic acid sequence of any one of SEQ ID NOs:2-7 and encoding a polypeptide according to SEQ ID NO:8, and having equivalent expression in a host to either expression of any one of SEQ ID NOs:2-7 or SEQ ID NO:1 expression, wherein the polynucleotide does not have the nucleic acid sequence of SEQ ID NO:1. In one embodiment, the synthetic polynucleotide has at least about 90% or at least about 95% or at least about 98% identity to the nucleic acid sequence of any one of SEQ ID NOs:2-7.
In one embodiment, the fragment includes only amino acid residues encoded by synPCCA, which represents the active, processed form of PCC alpha.

By active can be meant, for example, the enzyme's ability to catalyze the carboxylation of propionyl CoA to D-methylmalonyl CoA. The activity can be assayed using methods and assays well-known in the art (as described in the context of protein function, below).

In one embodiment of a synthetic polynucleotide according to the invention, the nucleic acid sequence encodes a polypeptide having the amino acid sequence of SEQ ID NO:8 or an amino acid sequence with at least about 90% identity to the amino acid sequence of SEQ ID NO:8.

In one embodiment, the synthetic polynucleotide exhibits augmented expression relative to the expression of naturally occurring human propionyl-CoA carboxylase alpha polynucleotide sequence (SEQ ID NO:1) in a subject. In yet another embodiment, the synthetic polynucleotide having augmented expression comprises a nucleic acid sequence comprising codons that have been optimized relative to the naturally occurring human propionyl-CoA carboxylase alpha polynucleotide sequence (SEQ ID NO:1). In still another embodiment of a synthetic polynucleotide according to the invention, the nucleic acid sequence has at least about 80% of less commonly used codons replaced with more commonly used codons.

In one embodiment of a synthetic polynucleotide according to the invention, the polynucleotide is a polynucleotide having a nucleic acid sequence with at least about 85% identity to the nucleic acid sequence of any one of SEQ ID NOs: 2-7. In other embodiments, the polynucleotide is a polynucleotide having a nucleic acid sequence with at least about 90% or 95% or 98% identity to the nucleic acid sequence of any one of SEQ ID NOs: 2-7.

In one embodiment of a synthetic polynucleotide according to the invention, the nucleic acid sequence is a DNA sequence. In one embodiment, the nucleic acid sequence is a RNA sequence or peptide modified nucleic acid sequence. In one embodiment, the synthetic polynucleotide according to the invention encodes an active PCC alpha fragment.

In another aspect, the invention is directed to an expression vector comprising the herein-described synthetic polynucleotide. In another embodiment of a vector according to the invention, the synthetic polynucleotide is operably linked to an expression control sequence. In still another embodiment, the synthetic polynucleotide is codon-optimized.

In one embodiment, the expression vector comprising a synthetic polynucleotide is an AAV vector containing the chicken-beta actin promoter (SEQ ID NO:9), the elongation factor 1 alpha long promoter (EF1AL) (SEQ ID NO:10), the elongation factor 1 alpha short promoter with a 3′ hepatitis B post translation response element (HPRE) (SEQ ID NO:11), or the short elongation factor 1 alpha promoter with a mutant 3′ hepatitis B post translation response element (HPRE) (SEQ ID NO:12).

In another embodiment, the expression vector comprising the synthetic PCCA polynucleotide is an AAV vector containing a liver specific enhancer and promoter, such as the long (SEQ ID NO:14) or short variants (SEQ ID NO:13) of the apolipoprotein E enhancer, operably linked to the long (SEQ ID NO:16) or short variants of the human alpha 1 antitrypsin promoter (SEQ ID NO:15) and followed by either a chimeric intron (SEQ ID NO:17), modified beta (β)-globin intron (SEQ ID NO: 18), or a synthetic intron (SEQ ID NO:19).

In one embodiment, the apolipoprotein E enhancer, and human alpha 1 antitrypsin promoter are operably linked to form a short (SEQ ID NO: 20) or long liver specific enhancer-promoter units (SEQ ID NO: 21) and placed 5′ to an intron selected from SEQ ID NO: 17-19. In one embodiment, the intron is the modified β-globin intron (SEQ ID NO: 18).

In a further aspect, the enhanced human alpha 1 antitrypsin enhancer, promoter, and intron comprises SEQ ID:22.

In another embodiment, the liver specific enhancer is derived from sequences upstream of the alpha-1-microglobulin/bikunin precursor (SEQ ID:23 and SEQ ID:24), operably linked to the human thyroxine-binding globulin promoter (TBG) (SEQ ID:25).

In one embodiment, the liver specific enhancer and human thyroxine-binding globulin promoter is SEQ ID:26.

The synthetic PCCA genes of the disclosure can include additional features. For example, the synthetic PCCA genes can be flanked by a 5′ untranslated region (5′UTR) that includes a strong Kozak translational initiation signal. A 5′UTR can comprise a heterologous polynucleotide fragment and a then a second, third or fourth polynucleotide fragment from the same and/or different UTRs.

In some embodiments, the polynucleotide of the disclosure comprises an internal ribosome entry site (IRES) (SEQ ID: 27) instead of, or in addition to, a UTR.

In one embodiment, the UTR can also include at least one translation enhancer element (TEE). A TEE comprises nucleic acid sequences that increase the amount of polypeptide or protein produced from a polynucleotide. As a non-limiting example, the TEE can be located between the promoter and the start codon. In some embodiments, the 5′UTR comprises a TEE.

In one embodiment, the 5′UTR sequence(s) are derived from genes well known to be highly expressed in the liver. Non-limiting examples include polynucleotides derived from human albumin (SEQ ID: 28), SERPINA 1 (SEQ ID: 29), or SERPINA 3 (SEQ ID: 30).

In one embodiment, the synthetic PCCA genes of the disclosure includes additional features, including the incorporation of sequences designed to stabilize the synthetic PCCA mRNA. In one example, the sequence comprises the wood chuck post-translational response element (SEQ ID: 31). In another non-limiting example, the sequence comprises the hepatitis post-translational response element (SEQ ID:32).

In one embodiment, an expression cassette is included containing synthetic PCCA includes a polyadenylation signal, such as that derived from the rabbit beta globin gene or the bovine growth hormone gene. Such sequences are well known to practitioners of the art.

In one embodiment, terminal repeat sequences (SEQ ID:33-34) from the piggyBac transposon, which is originally isolated from the cabbage looper (Trichoplusia ni; a moth species), are inserted immediately after the 5′AAV ITR and before the 3′ AAV ITR. piggyBac is a class II transposon, moving in a cut-and-paste manner. An AAV vector that contains piggyBac terminal repeat sequences can serve as a substrate for piggyBac transposase, which, when introduced by a viral or non-viral vector, can mediate the permanent integration of the AAV cassette containing synthetic PCCA into the transduced cell. Hybrid AAV-piggyBac transposon vectors are well understood by practitioners of the art, and can be used to deliver synthetic PCCA to a target cell in vitro and in vivo.

One embodiment of a AAV vector plasmid designed to express synPCCA1 incorporates the enhanced TBG promoter is SEQ ID:35.

In one embodiment, a AAV vector designed to express synPCCA1 incorporates the enhanced human alpha 1 antitrypsin promoter is SEQ ID:36.

In one embodiment, the synthetic PCCA genes are configured to integrate into the human albumin locus. A donor cassette is constructed that targets the stop codon of human albumin, which yields, after homologous recombination, synPCCA1 that is fused via a P2 peptide to the carboxy terminus of albumin.

In one embodiment, the vector is an integrating AAV vector, from 5′ITR to 3′ITR, that uses homologous recombination to insert synPCCA1 into end of Albumin, which is a safe harbor for gene editing, is SEQ ID:37.

In one embodiment, the integrating AAV vector, from 5′ITR to 3′ITR, that uses homologous recombination to insert synPCCA1 into 5′ end of Albumin is SEQ ID:38.

In one embodiment, the synthetic PCCA genes of this application is configured to integrate into the genome after delivery using a lentiviral vector.

In one embodiment, a lentiviral vector is designed to express synPCCA1 using an enhanced human alpha 1 antitrypsin enhancer and promoter is SEQ ID:39.

In yet another embodiment, a lentiviral vector designed to express synPCCA1 using the elongation factor 1 long promoter is SEQ ID:40.

In one embodiment, the invention is directed to a method of treating a disease or condition mediated by propionyl-CoA carboxylase or low levels of propionyl-CoA carboxylase activity, the method comprising administering to a subject the herein-described synthetic polynucleotide.

In one embodiment, the invention is directed to a method of treating a disease or condition mediated by propionyl-CoA carboxylase, the method comprising administering to a subject a propionyl-CoA carboxylase produced using the synthetic polynucleotide described herein. In another embodiment of a method of treatment according to the invention, the disease or condition is propionic acidemia (PA).

In one aspect, the invention is directed to a composition comprising the synthetic polynucleotide of claim 1 and a pharmaceutically acceptable carrier.

In one aspect, the invention is directed to a transgenic animal whose genome comprises a polynucleotide sequence encoding propionyl-CoA carboxylase alpha or a functional fragment thereof. In still another aspect, the invention is directed to a method for producing such a transgenic animal, comprising: providing an exogenous expression vector comprising a polynucleotide comprising a promoter operably linked to a polynucleotide encoding propionyl-CoA carboxylase alpha or a functional fragment thereof; introducing the vector into a fertilized oocyte; and transplanting the oocyte into a female animal.

In one aspect, the invention is directed to a transgenic animal whose genome comprises the synthetic polynucleotide described herein. In another aspect, the invention is directed to a method for producing such a transgenic animal, comprising: providing an exogenous expression vector comprising a polynucleotide comprising a promoter operably linked to the synthetic polynucleotide described herein; introducing the vector into a fertilized oocyte; and transplanting the oocyte into a female animal.

Methods for producing transgenic animals are known in the art and include, without limitation, transforming embryonic stem cells in tissue culture, injecting the transgene into the pronucleus of a fertilized animal egg (DNA microinjection), genetic/genome engineering, viral delivery (for example, retrovirus-mediated gene transfer).

Transgenic animals according to the invention include, without limitation, rodent (mouse, rat, squirrel, guinea pig, hamster, beaver, porcupine), frog, ferret, rabbit, chicken, pig, sheep, goat, cow primate, and the like.

In one aspect, the invention is directed to the preclinical amelioration or rescue from the disease state, for example, propionic acidemia, that the afflicted subject exhibits. This may include symptoms, such as lethargy, lethality, metabolic acidosis, and biochemical perturbations, such as increased levels of methylcitrate in blood, urine, and body fluids.

In one aspect, the invention is directed to a method for producing a genetically engineered animal as a source of recombinant synPCCA. In one aspect, genome editing, or genome editing with engineered nucleases (GEEN) may be performed with the synPCCA nucleotides of the present invention allowing synPCCA DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the nucleotides of the present invention including synPCCA, in combination with a CASP/CRISPR, ZFN, TALEN, or transposon such as piggyBac can be used to engineer correction at the locus in a patient's cell either in vivo or ex vivo, then, in one embodiment, use that corrected cell, such as a fibroblast or lymphoblast, to create an iPS or other stem cell for use in cellular therapy.

In one embodiment the synthetic polynucleotide having increased expression comprises a nucleic acid sequence comprising codons that have been optimized relative to the naturally occurring human propionyl-CoA carboxylase subunit a polynucleotide sequence (SEQ ID NO:1). In one embodiment, the nucleic acid sequence has at least about 70% of less commonly used codons replaced with more commonly used codons.

In one embodiment, the recombinant vector is a recombinant adeno-associated virus (rAAV), said rAAV comprising an AAV capsid, and a vector genome packaged therein, said vector genome comprising: a 5′-inverted terminal repeat sequence (5′-ITR) sequence; a promoter sequence; a 5′ untranslated region; a Kozak sequence; a partial fragment or complete coding sequence for PCCA; an mRNA stability sequence; a polyadenylation signal; and a 3′-inverted terminal repeat sequence (3′-ITR) sequence. In one embodiment, the rAAV is comprised of the structure in FIG. 9A. In one embodiment, the AAV capsid is from an AAV of serotype 1, 2, 3, 4, 5, 6, 7, 8, 9, 10, 11, 12, 13, rh 10, hu37 or Anc, and mutants thereof. In one embodiment, the AAV capsid is from an AAV of serotype 8. In one embodiment, the AAV capsid is from an AAV of serotype 9. In one embodiment, the rAAV further contains terminal repeat sequences recognized by piggyBac transposase internal to the 5′ and 3′ ITR.

In one embodiment, the promoter is selected from the group consisting of chicken-beta actin promoter (SEQ ID NO: 9), the elongation factor 1 alpha long promoter (EF1AL) (SEQ ID NO:10), the elongation factor 1 alpha short promoter with a 3′ hepatitis B post translation response element (HPRE) (SEQ ID NO:11), and the short elongation factor 1 alpha promoter with a mutant 3′ hepatitis B post translation response element (HPRE) (SEQ ID NO:12). In one embodiment, the promoter is selected from the group consisting of liver specific enhancer and promoter, such as the long (SEQ ID NO:14), or short variants (SEQ ID NO:13) of the apolipoprotein E enhancer, and further comprising operably linked to the long (SEQ ID NO:16) or short variants of the human alpha 1 antitrypsin promoter (SEQ ID NO:15), and optionally at least one intron selected from the group consisting of a chimeric intron (SEQ ID NO:17), modified β-globin intron (SEQ ID NO: 18), and a synthetic intron (SEQ ID NO:19). In one embodiment, the promoter is selected from the group consisting of a liver specific enhancer and promoters of a long (SEQ ID NO:14), or short variant (SEQ ID NO:13) of the apolipoprotein E enhancer, the enhanced human alpha 1 antitrypsin promoter (SEQ ID:36), and the enhanced TB G promoter (SEQ ID:35), further comprising operably linked to the long (SEQ ID NO:16) or short variants of the human alpha 1 antitrypsin promoter (SEQ ID NO:15) and followed by either a chimeric intron (SEQ ID NO:17), modified B-globin intron (SEQ ID NO: 18), or a synthetic intron (SEQ ID NO:19).

In one embodiment, the apolipoprotein E enhancer, and the human alpha 1 antitrypsin promoter are operably linked to form a short (SEQ ID NO: 20) or long liver specific enhancer-promoter units (SEQ ID NO: 21) and placed 5′ to an intron selected from SEQ ID NO:17-19. In one embodiment, the intron is the modified B-globin intron (SEQ ID NO: 18). In one embodiment, the intron comprises SEQ ID:22.

In one embodiment, the liver specific enhancer is derived from sequences upstream of the alpha-1-microglobulin/bikunin precursor (SEQ ID:23 and SEQ ID:24), and operably linked to the human thyroxine-binding globulin promoter (TBG) (SEQ ID:25). In one embodiment, the liver specific enhancer and human thyroxine-binding globulin promoter is SEQ ID:26.

In one embodiment, the synthetic PCCA gene is flanked by a 5′ untranslated region (5′UTR) that includes a strong Kozak translational initiation signal. A 5′UTR can comprise a heterologous polynucleotide fragment and a then a second, third or fourth polynucleotide fragment from the same and/or different UTRs. In one embodiment, the synthetic polynucleotide further comprises an internal ribosome entry site (IRES) (SEQ ID: 27) instead of, or in addition to, a UTR. In one embodiment, the synthetic polynucleotide further comprises at least one translation enhancer element (TEE). In one embodiment, the TEE is located between the promoter and the start codon. In one embodiment, the 5′UTR comprises a TEE. In one embodiment, the UTR comprises sequences selected from the group consisting of human albumin (SEQ ID: 28), SERPINA 1 (SEQ ID: 29), and SERPINA 3 (SEQ ID: 30).

In one embodiment, the polynucleotide further comprises the wood chuck post-translational response element (SEQ ID: 31) or the sequence comprises the hepatitis post-translational response element (SEQ ID:32).

In one embodiment, the synthetic polynucleotide further comprises a polyadenylation signal. In one embodiment, the polyadenylation signal is a rabbit beta globin gene or the bovine growth hormone gene.

In one embodiment, the rAAV further comprises terminal repeat sequences (SEQ ID: 33-34) from the piggyBac transposon, located after the 5′ AAV ITR and before the 3′ AAV ITR. piggyBac is a class II transposon.

In one embodiment, the synthetic polynucleotide further comprises a donor cassette that targets the stop codon of human albumin, which yields, after homologous recombination synPCCA1 fused via a P2 peptide to the carboxy terminus of albumin. In one embodiment, the synthetic polynucleotide further comprising an integrating AAV vector, from 5′ ITR to 3′ ITR, that uses homologous recombination to insert synPCCA1 into end of human Albumin, having a safe harbor for gene editing, is SEQ ID:37.

In one embodiment, the synthetic polynucleotide further comprises an AAV vector, from 5′ITR to 3′ITR, that relies upon homologous recombination to insert synPCCA1 into 5′ end of Albumin is SEQ ID:38.

In one embodiment, the synthetic PCCA gene is configured to integrate into the genome after delivery using a lentiviral vector.

In one embodiment, the lentiviral vector further comprises an enhanced human alpha 1 antitrypsin enhancer, promoter is SEQ ID:39. In one embodiment, the lentiviral vector further comprises the elongation factor 1 long promoter is SEQ ID:40.

In one embodiment, the promotor is a tissue specific promoter. In one embodiment, the tissue specific promotor is selected from the group consisting of Apo A-I, ApoE, hAAT, transthyretin, liver-enriched activator, albumin, TBG, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid)

In one embodiment, the expression vector is AAV2/9-CBA-synPCCA1. In one embodiment, the expression vector is AAV2/9-EF1L-synPCCA1. In one embodiment, the expression vector is AAV2/9-EF1S-HPRE synPCCA1. In one embodiment, the expression vector is AAV2/9-EF1S-mHPRE synPCCA1.

In one embodiment, a composition comprises the synthetic polynucleotide and a pharmaceutically acceptable carrier. In one embodiment, the composition comprises the expression vector and a pharmaceutically acceptable carrier. In one embodiment, the composition further comprises a hybrid AAV-piggyBac transposon system.

In one embodiment a method of treating a disease or condition mediated by propionyl-CoA carboxylase, comprises administering to a subject in need thereof a therapeutic amount of the synthetic polynucleotide. In one embodiment, the method comprises administering to a subject a propionyl-CoA carboxylase produced using the synthetic polynucleotide as described herein. In one embodiment, the disease or condition is propionic acidemia (PA).

In one embodiment, the method of treating a disease or condition mediated by propionic acidemia (PA), comprises administering to a cell of a subject in need thereof the polynucleotide of claim 1, wherein the polynucleotide is inserted into the cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system) and meganuclease re-engineered homing endonucleases on a cell from the subject; and administering the cell to the subject.

In one embodiment, the composition is administered subcutaneously, intramuscularly, intradermally, intraperitoneally, or intravenously.

In one embodiment, the rAAV is administered at a dose of about 1×10¹¹ to about 1×10¹⁴ genome copies (GC)/kg.

In one embodiment, the administering the rAAV comprises administration of a single dose of rAAV; in one embodiment, administering the rAAV comprises administration of a multiple doses of rAAV.

### BRIEF DESCRIPTION OF THE DRAWINGS

FIG. 1A presents the ClustalW weighted sequence distances and percent sequence identity of different PCCA alleles versus wild type PCCA, and each other, showing that all the synPCCA sequences (SEQ ID NOs: 2-7) differ from the wild type PCCA gene (SEQ ID NO: 1) by >20% at the nucleotide level, and similarly, diverge from each other between 11-24%.

FIG. 1B shows the characterization of distinct feature of the synPCCA sequences (SEQ ID NOs: 2-7) and the wild type PCCA gene (SEQ ID NO: 1) using a phylogenetic analysis where distinct grouping is apparent.

FIG. 2 presents a western blot showing PCCA protein expression in 293 cells, which are human transformed kidney cells, transfected with AAV backbones expressing GFP or either wild-type or synPCCA under the control of various promoter/enhancer combinations. PCCA=propionyl-CoA carboxylase alpha subunit, CBA=chicken beta actin, EF1a=elongation factor 1 alpha, EF1aS=elongation factor 1 alpha short.

FIG. 3 presents synPCCA directed PCCA protein levels relative to wild-type PCCA expression in transfected 293 cells, quantified from the western blot in FIG. 2. The PCCA expression is much higher in 293 cells transfected with CBA-synPCCA1 versus those transfected with CBA-PCCA (wild-type). The levels of CBA-PCCA (wild-type) are comparable to the expression achieved when using a weaker promoter and distinct synPCCA6 allele, EF1a-synPCCA6.

FIG. 4 Survival in untreated Pcca^{-/-} (n=12) mice compared to Pcca^{-/-} mice (n=4) treated with 3×10¹¹ VC of AAV-CBA-synPCCA1 delivered by intrahepatic injection at birth. Treated Pcca^{-/-} mice display a significant increase in survival and were indistinguishable from their wild-type litter mates. shows percent survival of untreated Pcca^{-/-} (n=10) mice compared to Pcca^{-/-} mice (n=9) treated with 3×10¹¹ VC of AAV-CBA-synPCCA1 delivered by systemic injection at birth. Treated Pcca^{-/-} mice display a significant increase in survival with some mice surviving for greater than 150 day and were indistinguishable from their wild-type litter mates, on day 30 of life.

FIG. 5 shows plasma methylcitrate levels in untreated Pcca^{-/-} (n=6) mice and Pcca^{-/-} mice (n=6) treated with 3×10¹¹ VC of AAV-CBA-synPCCA1 by systemic injection at birth. Treated Pcca^{-/-} mice have a significant decrease in the disease related biomarker, 2-methylcitrate.

FIG. 6 shows western blots of murine livers, from wild-type mice (Pcca^{+/+} and Pcca^{+/-}), an untreated propionic acidemia mouse (Pcca^{-/-}), and Pcca^{-/-} mouse treated with 3×10¹¹ VC of AAV9-CBA-synPCCA1. The AAV treated mouse was sacrificed on day of life 30 and injected on day of life 1. The treated Pcca^{-/-} mouse displays hepatic Pcca expression whereas the untreated Pcca^{-/-} mice shows no hepatic murine Pcca expression. The antibody used for western blot can detect both human (PCCA) and murine (Pcca).

FIG. 7 shows hepatic PCCA protein expression relative to wild-type murine PCCA expression in untreated and the AAV9 treated Pcca^{-/-} mouse quantified from western blot in FIG. 6.

FIG. 8. Survival in untreated Pcca^{-/-} (n=10) mice compared to Pcca^{-/-} mice (n=9) treated with 3×10¹¹ VC of AAV-CBA-synPCCA1 delivered by systemic injection at birth. Treated Pcca^{-/-} mice display a significant increase in survival and some treated mice were indistinguishable from their wild-type litter mates at day 30 and demonstrated long term survival to >150 days.

FIG. 9A shows a vector comprised of 145 base pair AAV2 inverted terminal repeats (5′ITR_{L} and 3′ ITR_{L}), the long elongation factor 1α promoter (EF1AL), an intron (I), the synPCCA1 gene, the rabbit beta-globin polyadenylation signal (rBGA). The production plasmid expresses the kanamycin resistance gene. FIG. 9B shows a vector comprised of 130 base pair AAV2 inverted terminal repeats (5′ITR_{S} and 3′ ITR_{S}), the short elongation factor 1α promoter (EF1AS), an intron (I), synPCCA1 gene, the hepatitis B post translation response element (HPRE), and the bovine growth hormone polyadenylation signal (BGHA). The production plasmid expresses the kanamycin resistance gene.

FIG. 10 presents a western blot showing PCCA protein expression in 293 cells, which are human transformed kidney cells, after transfection with transfected with AAV backbones expressing synPCCA1 under the control of various promoter/enhancer combinations. PCCA=propionyl-CoA carboxylase alpha subunit, CBA=chicken beta actin, EF1a=elongation factor 1 alpha, EF1aS=elongation factor 1 alpha short. HPRE—hepatitis B post translation response element. HPREm—hepatitis B post translation response element, mutant. Beta-actin is the loading control. The fold change of protein expression compared to the basal level in 293T cells in indicated above as fold change.

FIG. 11 depicts survival in untreated Pcca (n=12) mice compared to Pcca^{-/-} mice (n=9) treated with 1×10¹¹ VC of AAV9-EF1aL-synPCCA1 (n=18), 1×10¹¹ VC of AAV9-EF1aS-synPCCA1-HPRE (n=15), or 4×10¹¹ VC of AAV9-EF1aS-synPCCA1-HPRE (n=5) delivered by retroorbital injection at birth. The treated Pcca^{-/-} mice display a significant increase in survival, with many mice remaining alive at the time of this application.

FIG. 12 shows the list of codon frequencies in the human proteome.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Definitions

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices, and materials are now described.

As used in this application, including the appended claims, the singular forms “a,” “an,” and “the” include plural references, unless the content clearly dictates otherwise, and are used interchangeably with “at least one” and “one or more.” Thus, reference to “a polynucleotide” includes a plurality of polynucleotides or genes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

In the context of synPCCA, the terms “gene” and “transgene” are used interchangeably. A “transgene” is a gene that has been transferred from one organism to another.

The term “subject”, as used herein, refers to a domesticated animal, a farm animal, a primate, a mammal, for example, a human.

The phrase “substantially identical”, as used herein, refers to an amino acid sequence exhibiting high identity with a reference amino acid sequence (for example, at least 80%, at least 85%, at least 90%, at least 95%, at least 98%, or at least 99% identity) and retaining the biological activity of interest (the enzyme activity).

The polynucleotide sequences encoding the alpha subunit of PCC, synPCCA, allow for increased expression of the synPCCA gene relative to naturally occurring human PCCA sequences. These polynucleotide sequences are designed to not alter the naturally occurring human PCC alpha subunit amino acid sequence. They are also engineered or optimized to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. Because the sequences are novel, they facilitate detection using nucleic acid-based assays.

As used herein, “PCCA” refers to the alpha subunit of human propionyl-CoA carboxylase, and “Pcca” refers to the alpha subunit of mouse propionyl-CoA carboxylase. Propionyl-CoA carboxylase (PCC) catalyzes the carboxylation of propionyl-CoA to D-methylmalonyl-CoA which is a metabolic precursor to succinyl-CoA, a component of the citric acid cycle or tricarboxylic acid cycle (TCA). The genes encoding the alpha and beta subunits of naturally occurring human propionyl-CoA carboxylase gene are referred to as PCCA or PCCB, respectively. The synthetic polynucleotide encoding the alpha subunit of PCC is known as synPCCA.

Naturally occurring human propionyl-CoA carboxylase is referred to as PCC, while synthetic PCC is designated as synPCC, even though the two are identical at the amino acid level.

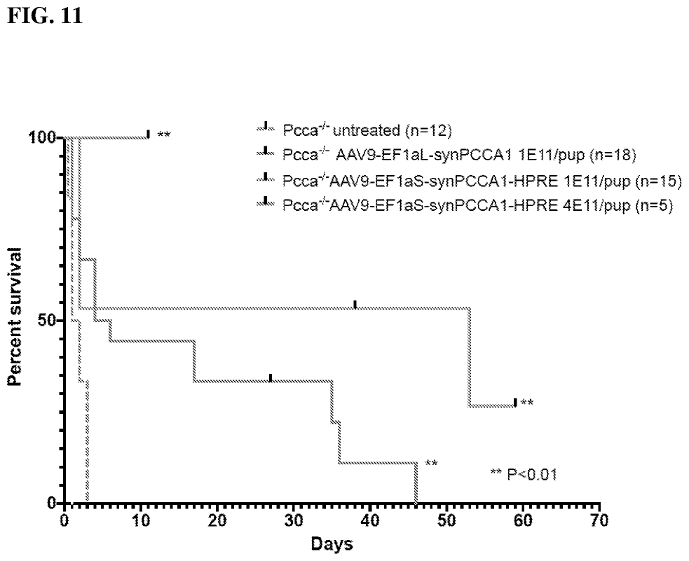

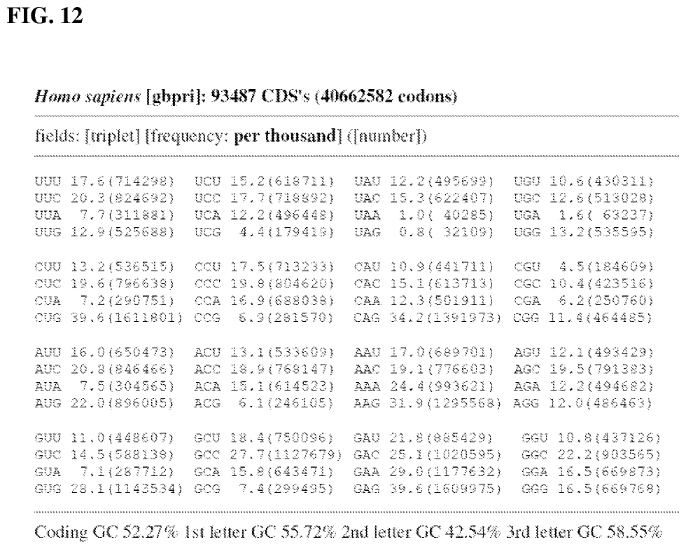

“Codon optimization” refers to the process of altering a naturally occurring polynucleotide sequence to enhance expression in the target organism, e.g., humans. In the subject application, the human PCCA gene has been altered to replace codons that occur less frequently in human genes with those that occur more frequently and/or with codons that are frequently found in highly expressed human genes, see FIG. 11.

As used herein, “determining”, “determination”, “detecting”, or the like are used interchangeably herein and refer to the detecting or quantitation (measurement) of a molecule using any suitable method, including immunohistochemistry, fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and its variations refer to the identification or observation of the presence of a molecule in a biological sample, and/or to the measurement of the molecule's value.

As used herein, a “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In certain embodiments, it may be preferable to include isotonic agents, for example, sugars, polyalcohols such as mannitol, sorbitol, or sodium chloride in the composition.

A “therapeutically effective amount” refers to an amount effective, at dosages and for periods of time necessary, to achieve the desired therapeutic result. A therapeutically effective amount of a vector comprising the synthetic polynucleotide of the invention may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the synthetic polynucleotide or a fragment thereof according to the invention calculated to produce the desired therapeutic effect in association with a pharmaceutical carrier.

### Additional Embodiments of the Invention

### The Synthetic Polynucleotide

In one embodiment of the invention, codon optimization was employed to create six highly active and synthetic PCCA alleles designated PCCA1-6. This method involves determining the relative frequency of a codon in the protein-encoding genes in the human genome. For example, isoleucine can be encoded by AUU, AUC, or AUA, but in the human genome, AUC (47%), AUU (36%), and AUA (17%) are variably used to encode isoleucine in proteins. Therefore, in the proper sequence context, AUA would be changed to AUC to allow this codon to be more efficiently translated in human cells. FIG. 11 presents the codon usage statistics for a large fraction of human protein-encoding genes and serves as the basis for changing the codons throughout the PCCA cDNA.

Thus, the invention comprises synthetic polynucleotides encoding propionyl-CoA carboxylase subunit alpha (PCCA) selected from the group consisting of SEQ ID NOs: 2-7 and a polynucleotide sequence having at least about 80% identity thereto. For those polynucleotides having at least about 80% identity to SEQ ID NOs: 2-7, in additional embodiments, they have at least 85%, at least 90%, at least 95%, at least 97%, at least 98%, or at least 99% identity.

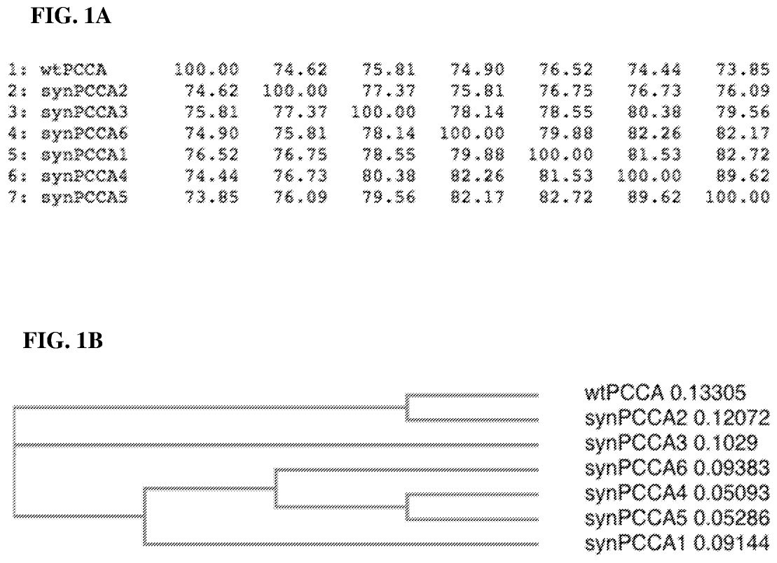

In one embodiment, the subject synthetic polynucleotide encodes a polypeptide with 100% identity to the naturally occurring human PCC protein. FIG. 1A presents the ClustalW weighted sequence distances and percent sequence identity of different PCCA alleles versus wild type PCCA, and each other, showing that all the synPCCA sequences (SEQ ID NOs: 2-7) differ from the wild type PCCA gene (SEQ ID NO: 1) by >20% at the nucleotide level, and similarly, diverge from each other between 11-24%. FIG. 1B shows the characterization of distinct feature of the synPCCA sequences (SEQ ID NOs: 2-7) and the wild type PCCA gene (SEQ ID NO: 1) using a phylogenetic analysis where distinct grouping is apparent.

TABLE 1
Sequences of wild-type and codon-optimized (or syn) PCCA alleles

| PCCA Allele | Sequences |
|---|---|
| wtPCCA | SEQ ID NO: 1 |
| synPCCA1 | SEQ ID NO: 2 |
| synPCCA2 | SEQ ID NO: 3 |
| synPCCA3 | SEQ ID NO: 4 |
| synPCCA4 | SEQ ID NO: 5 |
| synPCCA5 | SEQ ID NO: 6 |
| synPCCA6 | SEQ ID NO: 7 |

TABLE 2
Sequence alignment of the synthetic PCCA alleles compared to each
other and the wild type PCCA sequence using CLUSTAL multiple
sequence alignment by MUSCLE (3.8)

| | |
|---|---|
| wtPCCA | ATGGCGGGGTTCTGGGTCGGGACAGCACCGCTGGTCGCTGCCGGACGGCGTGGGCGGTGG |
| synPCCA2 | ATGGCCGGGTTTTGGGTGGGCACGGCCCCGCTCGTAGCAGCTGGCAGGCGGGGGCGATGG |
| synPCCA3 | ATGGCCGGCTTCTGGGTGGGGACTGCTCCCCTTGTCGCCGCAGGACGCAGAGGCCGCTGG |
| synPCCA6 | ATGGCCGGATTTTGGGTCGGAACTGCACCACTTGTCGCTGCCGGTAGAAGAGGAAGATGG |
| synPCCA1 | ------------------------------------------------------------ |
| synPCCA4 | ATGGCCGGATTTTGGGTTGGAACAGCTCCTCTGGTGGCCGCTGGGAGAAGAGGAAGATGG |
| synPCCA5 | ATGGCCGGCTTCTGGGTGGGCACCGCCCCCCTGGTGGCCGCCGGCAGAAGAGGCAGATGG |
| wtPCCA | CCGCCGCAGCAGCTGATGCTGAGCGCGGCGCTGCGGACCCTGAAGCATGTTCTGTACTAT |
| synPCCA2 | CCCCCCCACCAGCTTATGCTTAGTGCCGCCTTGCGGACGCTGAAGCACGTCCTTTACTAC |
| synPCCA3 | CCTCCTCACCACCTCATGCTCTCAGCAGCTCTGAGGACCCTGAAACACGTGCTTTACTAC |
| synPCCA6 | CCACCGCACCAACTGATGTTGAGCGCTGCACTGCGCACACTGAAGCATGTGCTGTACTAC |
| synPCCA1 | ---------------ATGCTGAGCGCAGCCCTGAGGACCCTGAAGCACGTGCTGTACTAT |
| synPCCA4 | CCTCCTCACCACCTGATGCTGTCTGCCGCTCTGAGAACCCTGAAACACGTGCTGTACTAC |
| synPCCA5 | CCCCCCCACCAGCTGATGCTGAGCGCCGCCCTGAGAACCCTGAAGCACGTGCTGTACTAC |
| | *** * ** ** ** * ** ***** ** ** ** ***** |
| wtPCCA | TCAAGACAGTGCTTAATGGTGTCCCGTAATCTTGGTTCAGTGGGATATGATCCTAATGAA |
| synPCCA2 | TCTAGACAGTGCCTTATGGTAAGCCGAAATTTGGGAAGTGTAGGTTATGATCCCAACGAG |
| synPCCA3 | AGTCGACAGTGTCTGATGGTGTCTAGGAACCTGGGTAGCGTGGGCTATGATCCCAATGAA |
| synPCCA6 | TCGCGCCAGTGTTTGATGGTGTCCAGGAATCTCGGCTCCGTGGGCTACGACCCCAACGAA |
| synPCCA1 | TCTAGGCAGTGCCTGATGGTCAGCCGCAACCTGGGCAGCGTGGGATACGACCCTAATGAG |
| synPCCA4 | AGCCGGCAGTGCCTGATGGTGTCCAGAAATCTGGGCAGCGTGGGCTACGACCCCAACGAG |
| synPCCA5 | AGCAGACAGTGCCTGATGGTGAGCAGAAACCTGGGCAGCGTGGGCTACGACCCCAACGAG |
| | * ***** * ***** * ** * ** ** ** ** ** ** ** ** |
| wtPCCA | AAAACTTTTGATAAAATTCTTGTTGCTAATAGAGGAGAAATTGCATGTCGGGTTATTAGA |
| synPCCA2 | AAGACCTTTGATAAGATACTGGTTGCTAACCGAGGGGAGATAGCGTGTCGAGTTATTCGC |
| synPCCA3 | AAGACCTTTGACAAAATACTGGTCGCTAATAGAGGGGAAATTGCTTGTCGCGTGATACGG |
| synPCCA6 | AAGACTTTTGACAAGATCCTCGTGGCCAACAGAGGGGAAATTGCGTGCCGCGTGATTCGG |
| synPCCA1 | AAGACATTCGATAAAATCCTGGTGGCTAACCGCGGCGAAATCGCATGCCGAGTGATTCGG |
| synPCCA4 | AAAACCTTCGACAAGATCCTGGTGGCCAACCGGGGAGAGATCGCCTGCAGAGTGATCCGG |
| synPCCA5 | AAGACCTTCGACAAGATCCTGGTGGCCAACAGAGGCGAGATCGCCTGCAGAGTGATCAGA |
| | ** ** ** ** ** ** ** ** ** ** * ** ** ** ** ** * ** ** * |
| wtPCCA | ACTTGCAAGAAGATGGGCATTAAGACAGTTGCCATCCACAGTGATGTTGATGCTAGTTCT |
| synPCCA2 | ACCTGTAAGAAGATGGGAATTAAAACCGTGGCCATCCATAGCGATGTCGACGCTTCCAGT |
| synPCCA3 | ACGTGCAAGAAGATGGGTATCAAAACCGTGGCAATTCACTCTGACGTTGATGCTTCCTCA |
| synPCCA6 | ACTTGCAAGAAGATGGGAATCAAGACCGTGGCCATACACTCCGATGTGGACGCCTCCTCC |
| synPCCA1 | ACCTGTAAGAAAATGGGGATCAAGACAGTCGCCATTCACAGCAGCGTGGATGCCAGCAGC |
| synPCCA4 | ACCTGCAAGAAGATGGGCATCAAGACCGTGGCCATCCACTCCGATGTGGATGCCTCTAGC |
| synPCCA5 | ACCTGCAAGAAGATGGGCATCAAGACCGTGGCCATCCACAGCGACGTGGACGCCAGCAGC |
| | ** ** ***** ***** ** ** ** ** ** ** ** ** ** ** ** |
| wtPCCA | GTTCATGTGAAAATGGCGGATGAGGCTGTCTGTGTTGGCCCAGCTCCCACCAGTAAAAGC |
| synPCCA2 | GTGCACGTTAAAATGGCCGACGAGGCCGTATGCGTGGGGCCTGCCCCTACCTCTAAGTCA |
| synPCCA3 | GTGCATGTAAAGATGGCGGATGAGGCTGTTTGCGTGGGTCCAGCACCTACAAGCAAGAGC |
| synPCCA6 | GTCCACGTCAAGATGGCTGATGAAGCCGTCTGCGTGGGACCGGCGCCTACTTCCAAGTCG |
| synPCCA1 | GTCCATGTGAAGATGGCAGACGAGGCCGTCTGCGTGGGACCAGCCCCTACATCTAAAAGT |
| synPCCA4 | GTGCACGTGAAAATGGCCGATGAGGCCGTGTGTGTGGGCCCTGCTCCTACAAGCAAGAGC |
| synPCCA5 | GTGCACGTGAAGATGGCCGACGAGGCCGTGTGCGTGGGCCCCGCCCCCACCAGCAAGAGC |
| | ** ** ** ** ***** ** ** ** ** ** ** ** ** ** ** ** ** |
| wtPCCA | TACCTCAACATGGATGCCATCATGGAAGCCATTAAGAAAACCAGGGCCCAAGCTGTACAT |
| synPCCA2 | TACCTGAACATGGATGCAATTATGGAAGCTATTAAGAAGACTCGGGCGCAGGCTGTCCAC |
| synPCCA3 | TATCTCAACATGGATGCCATCATGGAAGCTATCAAGAAAACCCGTGCACAAGCTGTGCAT |
| synPCCA6 | TACCTTAACATGGACGCCATCATGGAGGCCATCAAGAAAACCAGGGCGCAGGCGGTGCAT |
| synPCCA1 | TACCTGAACATGGATGCTATCATGGAAGCAATTAAGAAAACTAGGGCCCAGGCTGTGCAC |
| synPCCA4 | TACCTGAACATGGACGCCATCATGGAAGCCATTAAGAAAACAAGAGCCCAGGCCGTGCAT |
| synPCCA5 | TACCTGAACATGGACGCCATCATGGAGGCCATCAAGAAGACCAGAGCCCAGGCCGTGCAC |
| | ** ** ******** ** ** ***** ** ** ***** ** * ** ** ** ** ** |
| wtPCCA | CCAGGTTATGGATTCCTTTCAGAAAACAAAGAATTTGCCAGATGTTTGGCAGCAGAAGAT |
| synPCCA2 | CCTGGATATGGATTTCTTTCTGAGAATAAGGAGTTTGCCCGGTGTCTGGCGGCAGAAGAC |
| synPCCA3 | CCAGGGTATGGCTTTCTCTCCGAGAACAAAGAATTTGCCCGGTGTCTGGCAGCGGAGGAC |
| synPCCA6 | CCTGGCTACGGCTTCCTGTCCGAAAACAAGGAGTTCGCACGGTGCCTGGCCGCCGAGGAC |
| synPCCA1 | CCTGGCTATGGGTTCCTGAGCGAGAATAAGGAATTTGCACGATGTCTGGCAGCTGAGGAC |
| synPCCA4 | CCCGGCTACGGATTTCTGAGCGAGAACAAAGAATTTGCCCGGTGCCTGGCCGCCGAGGAC |
| synPCCA5 | CCCGGCTACGGCTTCCTGAGCGAGAACAAGGAGTTCGCCAGATGCCTGGCCGCCGAGGAC |
| | ** ** ** ** ** ** ** ** ** ** ** ** * ** **** ** ** ** |
| wtPCCA | GTCGTTTTCATTGGACCTGACACACATGCTATTCAAGCCATGGGCGACAAGATTGAAAGC |
| synPCCA2 | GTCGTATTCATTGGACCGGATACGCACGCTATCCAAGCCATGGGAGATAAGATCGAGAGC |
| synPCCA3 | GTGGTGTTCATTGGGCCTGATACGCATGCAATTCAAGCCATGGGCGATAAGATTGAGAGC |
| synPCCA6 | GTGGTCTTTATCGGGCCCGACACCCATGCAATCCAGGCCATGGGCGACAAGATCGAGTCG |
| synPCCA1 | GTGGTCTTTATCGGACCAGATACACATGCTATTCAGGCAATGGGCGACAAGATCGAGTCC |
| synPCCA4 | GTGGTGTTTATTGGCCCTGATACACACGCCATCCAGGCCATGGGCGATAAGATCGAGTCT |
| synPCCA5 | GTGGTGTTCATCGGCCCCGACACCCACGCCATCCAGGCCATGGGCGACAAGATCGAGAGC |
| | ** ** ** ** ** ** ** ** ** ** ** ** ** ***** ** ***** ** |
| wtPCCA | AAATTATTAGCTAAGAAAGCAGAGGTTAATACAATCCCTGGCTTTGATGGAGTAGTCAAG |
| synPCCA2 | AAGCTCCTGGCTAAGAAAGCTGAAGTGAACACCATTCCTGGCTTTGACGGCGTGGTGAAG |
| synPCCA3 | AAGCTGCTTGCTAAGAAAGCAGAAGTTAACACAATCCCAGGCTTTGACGGCGTTGTCAAA |
| synPCCA6 | AAGCTGCTGGCGAAGAAGGCAGAAGTGAACACTATTCCCGGGTTCGACGGAGTGGTCAAA |
| synPCCA1 | AAACTGCTGGCCAAGAAAGCTGAAGTGAATACTATCCCCGGGTTCGACGGAGTGGTCAAG |
| synPCCA4 | AAGCTGCTGGCCAAGAAAGCCGAAGTGAACACAATCCCCGGCTTCGACGGCGTGGTCAAG |
| synPCCA5 | AAGCTGCTGGCCAAGAAGGCCGAGGTGAACACCATCCCCGGCTTCGACGGCGTGGTGAAG |
| | ** * * ** ***** ** ** ** ** ** ** ** ** ** ** ** ** ** ** |
| wtPCCA | GATGCAGAAGAAGCTGTCAGAATTGCAAGGGAAATTGGCTACCCTGTCATGATCAAGGCC |
| synPCCA2 | GACGCAGAGGAAGCTGTTCGCATCGCCCGCGAAATTGGATATCCCGTGATGATAAAAGCA |
| synPCCA3 | GACGCCGAAGAAGCGGTACGTATTGCCCGAGAAATCGGCTACCCCGTTATGATCAAGGCG |
| synPCCA6 | GACGCGGAAGAGGCCGTCCGAATCGCCCGGGAGATTGGATACCCTGTGATGATTAAGGCC |
| synPCCA1 | GATGCAGAGGAAGCCGTGAGAATCGCCAGGGAGATTGGCTACCCTGTGATGATTAAGGCA |
| synPCCA4 | GATGCTGAAGAAGCCGRGCGGARCGCCAGAGAAATCGGCTACCCCGTGATGATCAAAGCC |
| synPCCA5 | GACGCCGAGGAGGCCGTGAGAATCGCCAGAGAGATCGGCTACCCCGTGATGATCAAGGCC |
| | ** ** ** ** ** ** * ** ** * ** ** ** ** ** ** ***** ** ** |
| wtPCCA | TCAGCAGGTGGTGGTGGGAAAGGCATGCGCATTGCTTGGGATGATGAAGAGACCAGGGAT |
| synPCCA2 | TCTGCGGGGGGGGGCGGGAAGGGCATGAGAATTGCCTGGGATGATGAAGAAACTAGAGAT |
| synPCCA3 | TCAGCCGGAGGTGGAGGAAAAGGGATGAGGATTGCCTGGGATGACGAGGAGACTAGGGAT |
| synPCCA6 | TCGGCTGGCGGAGGCGGAAAGGGAATGCGCATTGCCTGGGATGACGAAGAAACCCGGGAT |
| synPCCA1 | TCTGCCGGCGGGGGAGGCAAAGGGATGAGGATCGCCTGGGACGATGAGGAAACTCGCGAT |
| synPCCA4 | TCTGCTGGCGGAGGCGGCAAGGGAATGAGAATCGCCTGGGACGACGAAGAGACACGCGAC |
| synPCCA5 | AGCGCCGGCGGCGGCGGCAAGGGCATGAGAATCGCCTGGGACGACGAGGAGACCAGAGAC |
| | ** ** ** ** ** ** ** *** * ** ** ***** ** ** ** ** * ** |
| wtPCCA | GGTTTTAGATTGTCATCTCAAGAAGCTGCTTCTAGTTTTGGCGATGATAGACTACTAATA |
| synPCCA2 | GGTTTCCGCTTGTCTTCTCAGGAAGCCGCATCATCCTTTGGAGATGACCGATTGCTCATA |
| synPCCA3 | GGGTTCCGGCTCTCCAGTCAGGAAGCAGCATCTTCTTTTGGTGACGATAGACTGCTGATA |
| synPCCA6 | GGATTCCGGCTGAGCTCCCAAGAAGCCGCATCGTCCTTCGGGGACGATAGACTGCTGATC |
| synPCCA1 | GGATTTCGACTGTCTAGTCAGGAAGCAGCCAGCAGCTTCGGCGACGATAGGCTGCTGATC |
| synPCCA4 | GGCTTTAGACTGAGCAGCCAAGAAGCCGCCAGCTCCTTCGGAGATGACAGACTGCTGATC |
| synPCCA5 | GGCTTCAGACTGAGCAGCCAGGAGGCCGCCAGCAGCTTCGGCGACGACAGACTGCTGATC |
| | ** ** * * ** ** ** ** ** ** ** ** * * ** ** |
| wtPCCA | GAAAAATTTATTGATAATCCTCGTCATATAGAAATCCAGGTTCTAGGTGATAAACATGGG |
| synPCCA2 | GAGAAATTTATCGACAATCCACGGCATATTGAGATCCAAGTGCTTGGCGACAAGCACGGT |
| synPCCA3 | GAGAAATTCATCGACAACCCTCGACACATTGAAATCCAGGTACTGGGAGACAAACACGGA |
| synPCCA6 | GAAAAGTTCATCGACAACCCAAGGCACATCGAAATCCAGGTCCTCGGGGACAAGCATGGA |
| synPCCA1 | GAGAAGTTCATTGACAACCCCCGCCACATCGAAATTCAGGTGCTGGGGGATAAACATGGA |
| synPCCA4 | GAGAAGTTCATCGACAACCCCAGACACATCGAGATCCAGGTGCTGGGCGACAAGCACGGA |
| synPCCA5 | GAGAAGTTCATCGACAACCCCAGACACATCGAGATCCAGGTGCTGGGCGACAAGCACGGC |
| | ** ** ** ** ** ** ** * ** ** ** ** ** ** ** ** ** ** ** ** |
| wtPCCA | AATGCTTTATGGCTTAATGAAAGAGAGTGCTCAATTCAGAGAAGAAATCAGAAGGTGGTG |
| synPCCA2 | AACGCGCTTTGGCTCAACGAACGAGAGTGTTCAATCCAGAGGAGGAACCAGAAGGTTGTA |
| synPCCA3 | AATGCACTTTGGCTCAATGAACGCGAGTGCTCCATTCAGCGCAGGAACCAGAAAGTCGTC |
| synPCCA6 | AACGCCCTGTGGTTGAACGAGAGAGAGTGCTCCATTCAACGGCGCAACCAGAAGGTCGTG |
| synPCCA1 | AACGCCCTGTGGCTGAATGAGCGGGAATGTAGCATTCAGCGGAGAAATCAGAAGGTGGTC |
| synPCCA4 | AATGCCCTGTGGCTGAACGAGAGAGAGTGCAGCATCCAGCGGCGGAACCAGAAAGTGGTG |
| synPCCA5 | AACGCCCTGTGGCTGAACGAGAGAGAGTGCAGCATCCAGAGAAGAAACCAGAAGGTGGTG |
| | ** ** * *** * ** ** * ** ** ** ** * * ** ***** ** ** |
| wtPCCA | GAGGAAGCACCAAGCATTTTTTTGGATGCGGAGACTCGAAGAGCGATGGGAGAACAAGCT |
| synPCCA2 | GAAGAAGCACCATCTATTTTCCTCGACGCAGAAACTCGGCGGGCTATGGGGGAACAAGCA |
| synPCCA3 | GCGGAAGCACCCTCCATCTTCCTGGATGCCGAGACAAGGCGCGCTATGGGCGAGCAGGCC |
| synPCCA6 | GAGGAAGCCCCCTCGATTTTCCTCGATGCTGAAACTCGCCGGGCCATGGGGGAGCAAGCG |
| synPCCA1 | GAGGAAGCTCCTTCCATCTTTCTGGACGCCGAGACAAGGCGCGCTATGGGAGAACAGGCT |
| synPCCA4 | GAAGAGGCCCCTAGCATCTTCCTGGACGCCGAAACTCGGAGAGCCATGGGAGAACAGGCT |
| synPCCA5 | GAGGAGGCCCCCAGCATCTTCCTGGACGCCGAGACCAGAAGAGCCATGGGCGAGCAGGCC |
| | ** ** ** ** ** ** * ** ** ** ** * * ** ***** ** ** ** |
| wtPCCA | GTAGCTCTTGCCAGAGCAGTAAAATATTCCTCTGCTGGGACCGTGGAGTTCCTTGTGGAC |
| synPCCA2 | GTGGCACTGGCTCGAGCCGTTAAATATTCTAGTGCGGGGACAGTAGAATTCCTCGTAGAT |
| synPCCA3 | GTTGCACTCGCTAGAGCCGTGAAGTACTCTTCTGCGGGTACCGTGGAATTTCTGGTAGAC |
| synPCCA6 | GTGGCCCTGGCCCGCGCAGTGAAGTACTCCTCGGCCGGGACCGTGGAGTTCCTGGTGGAC |
| synPCCA1 | GTCGCACTGGCCAGAGCTGTGAAATACTCCTCTGCCGGCACTGTCGAGTTCCTGGTGGAC |
| synPCCA4 | GTGGCTCTGGCTAGAGCCGTGAAGTATAGCAGCGCCGGCACCGTGGAATTTCTGGTGGAC |
| synPCCA5 | GTGGCCCTGGCCAGAGCCGTGAAGTACAGCAGCGCCGGCACCGTGGAGTTCCTGGTGGAC |
| | ** ** ** ** * ** ** ** ** ** ** ** ** ** ** ** ** ** |
| wtPCCA | TCTAAGAAGAATTTTTATTTCTTGGAAATGAATACAAGACTCCAGGTTGAGCATCCTGTC |
| synPCCA2 | AGCAAGAAGAATTTTTATTTTCTTGAGATGAATACGCGCCTTCAAGTGGAACACCCAGTC |
| synPCCA3 | AGCAAGAAGAACTTCTATTTCCTGGAGATGAATACCCGGCTGCAAGTCGAGCATCCAGTC |
| synPCCA6 | AGCAAAAAGAACTTCTACTTTCTCGAGATGAACACCAGGCTCCAAGTGGAGCACCCTGTG |
| synPCCA1 | AGCAAGAAAAACTTCTATTTTCTGGAAATGAACACCCGGCTGCAGGTCGAGCACCCAGTG |
| synPCCA4 | AGCAAGAAGAACTTCTACTTCCTCGAGATGAACACCCGGCTGCAGGTCGAGCACCCTGTG |
| synPCCA5 | AGCAAGAAGAACTTCTACTTCCTGGAGATGAACACCAGACTGCAGGTGGAGCACCCCGTG |
| | ** ** ** ** ** ** * ** ***** ** * ** ** ** ** ** ** ** |
| wtPCCA | ACAGAATGCATTACTGGCCTGGACCTAGTCCAGGAAATGATCCGTGTTGCTAAGGGCTAC |
| synPCCA2 | ACGGAATGTATAACTGGCCTTGACTTGGTTCAGGAGATGATACGGGTGGCTAAGGGTTAT |
| synPCCA3 | ACTGAGTGTATAACTGGCCTGGACCTGGTACAGGAAATGATTCGTGTAGCGAAGGGATAC |
| synPCCA6 | ACCGAATGCATCACTGGACTTGACCTGGTGCAGGAAATGATCCGCGTGGCCAAGGGATAC |
| synPCCA1 | ACTGAATGCATTACCGGGCTGGATCTGGTCCAGGAGATGATCAGAGTGGCCAAGGGATAC |
| synPCCA4 | ACCGAGTGTATCACAGGCCTGGACCTGGTGCAAGAGATGATCAGAGTGGCCAAGGGCTAC |
| synPCCA5 | ACCGAGTGCATCACCGGCCTGGACCTGGTGCAGGAGATGATCAGAGTGGCCAAGGGCTAC |
| | ** ** ** ** ** ** ** ** * ** ** ** ***** * ** ** ***** ** |
| wtPCCA | CCTCTCAGGCACAAACAAGCTGATATTCGCATCAACGGCTGGGCAGTTGAATGTCGGGTT |
| synPCCA2 | CCTCTTCGGCATAAGCAGGCTGATATTCGCATAAATGGGTGGGCGGTCGAGTGCAGAGTT |
| synPCCA3 | CCGCTCCGGCACAPACAAGCCGACATTCGCATCAATGGGTGGGCTGTGGAGTGCAGAGTC |
| synP1CA6 | CCCCTGAGGCACAAGCAGGCCGACATCAGAATCAACGGTTGGGCCGTGGAATGTCGGGTG |
| synP1CA1 | CCCCTGCGACATAAACAGGCTGACATCCGGATTAACGGCTGGGCAGTCGAGTGTCGGGTG |
| synP1CA4 | CCTCTGAGACACAAGCAGGCCGACATCCGGATCAATGGCTGGGCCGTTGAGTGCAGAGTG |
| synPCCA5 | CCCCTGAGACACAAGCAGGCCGACATCAGAATCAACGGCTGGGCCGTGGAGTGCAGAGTG |
| | ** ** * ** ** ** ** ** ** * ** ** ** ***** ** ** ** * ** |
| wtPCCA | TATGCTGAGGACCCCTACAAGTCTTTTGGTTTACCATCTATTGGGAGATTGTCTCACTAC |
| synPCCA2 | TATGCTGAGGACCCATACAAGTCATTCGGACTTCCTTCTATAGGCAGACTGTCACAATAT |
| synPCCA3 | TATGCAGAGGATCCCTATAAGTCCTTCGGGCTTCCCTCCATAGGCAGGCTTAGTCACTAT |
| synPCCA6 | TACGCTGAGGATCCGTATAAGTCCTTCGGCTTGCCGAGCATCGGACGGCTGTCACACTAC |
| synPCCA1 | TACGCCGAAGATCCATATAAGTCTTTCGGACTGCCCAGTATTGGCCGACTGTCACACTAT |
| synPCCA4 | TACGCCGAGGATCCCTACAAGACCTTCGGCCTGCCTAGCATCGGCCGGCTGTCTCACTAT |
| synPCCA5 | TACGCCGAGGACCCCTACAAGACCTTCGGCCTGCCCAGCATCGGCAGACTGAGCCACTAC |
| | ** ** ** ** ** ** *** ** ** * ** ** ** * * ** ** |
| wtPCCA | CAAGAACCGTTACATCTACCTGGTGTCCGAGTGGACAGTCGCATCCAACCAGGAAGTGAT |
| synPCCA2 | CAAGAGCCACTTCATCTCCCAGGTGTAAGAGTAGATTCCGGAATACAACCTGGCTCCGAT |
| synPCCA3 | CAGGAGCCATTGCACTTGCCTGGCGTCAGGGTGGACTCCGGCATCCAACCGGGCAGCGAC |
| synPCCA6 | CAGGAACCCCTGCACCTTCCTGGAGTCAGAGTGGACTCCGGAATCCAACCTGGTTCGGAC |
| synPCCA1 | CAGGAGCCTCTGCACCTGCCAGGCGTCAGAGTGGACAGCGGCATCCAGCCTGGGTCCGAC |
| synPCCA4 | CAAGAGCCACTGCATCTGCCCGGCGTCAGAGTGGATTCTGGAATCCAGCCTGGCAGCGAC |
| synPCCA5 | CAGGAGCCCCTGCACCTGCCCGCCGTGAGAGTGGACAGCGGCATCCAGCCCGGCAGCGAC |
| | ** ** ** * ** * ** ** ** * ** ** ** ** ** ** ** ** |
| wtPCCA | ATTAGCATTTATTATGATCCTATGATTTCAAAACTAATCACATATGGCTCTGATAGAACT |
| synPCCA2 | ATATCTATTTACTATGATCCAATGATTAGTAAGTTGATTACATATGGGAGTGATCGGACC |
| synPCCA3 | ATTTCAATTTACTACGATCCCATGATCAGCAAGTTGATTACCTATGGATCTGACCGGACA |
| synPCCA6 | ATTTCCATCTACTACGATCCGATGATCTCCAAACTCATTACCTACGGTAGCGACCGGACC |
| synPCCA1 | ATCTCTATCTACTATGATCCAATGATCAGCAAGCTGATTACATACGGCTCCGATCGGACT |
| synPCCA4 | ATCAGCATCTACTACGACCCTATGATCTCCAAGCTGATCACCTACGGCAGCGACCGGACA |
| synPCCA5 | ATCAGCATCTACTACGACCCCATGATCACCAAGCTGATCACCTACGGCAGCGACAGAACC |
| | ** ** ** ** ** ** ***** ** * ** ** ** ** ** * ** |
| wtPCCA | GAGGCACTGAAGAGAATGCCACATGCACTGGATAACTATGTTATTCGAGGTGTTACACAT |
| synPCCA2 | GAAGCTTTGAAGCGGATGCCGCACGCGCTGGATAACTACGTGATAAGGGGTGTCACGCAC |
| synPCCA3 | GAGGCTCTGAAGAGAATGCCCCACGCCCTGGACAATTACGTGATAAGAGGAGTGACACAC |
| synPCCAG | GAGGCTCTGAAACGCATGCCTCACGCCCTGGACAACTATGTCATCCGGGGAGTCACTCAC |
| synPCCA1 | GAGGCCCTGALAAGAATGCCACACGCCCTGGATAACTATGTCATTAGAGGGGTGACCCAT |
| synPCCA4 | GAGGCCCTGAAGAGAATGCCTCACGCCCTGGACAACTACGTGATCAGAGGCGTGACCCAC |
| synPCCA5 | GAGGCCCTGAAGAGAATGCCCCACGCCCTGGACAACTACGTGATCAGAGGCGTGACCCAC |
| | ** ** **** * ***** ** ** ***** ** ** ** ** * ** ** ** ** |
| wtPCCA | AATATTGCATTACTTCGAGAGGTGATAAcCAACTCACGCTTTGTAAAAGGAGACATCAGC |
| synPCCA2 | AATATAGCTCTGCTGAGGGAGGTAATTATCAACAGTCGGTTCGTGAAGGGTGACATTAGC |
| synPCCA3 | AACATTGCCCTGTTGCGGGAGGTGATCATCAATAGCAGATTCGTGAAGGGTGACATCTCC |
| synPCCA6 | AATATCGCGCTGCTGCGCGAAGTCATCATTAATAGCCGCTTCGTGAAGGGCGACATTTCC |
| synPCCA1 | AATATCGCTCTGCTGAGAGAAGTCATCATTAACTCCAGGTTCGTGAAGGGAGACATCAGC |
| synPCCA4 | AATATCGCCCTGCTGCGGGAAGTGATCATCAACAGCAGATTCGTGAAAGGCGATATCAGC |
| synPCCA5 | AACATCGCCCTGCTGAGAGAGGTGATCATCAACAGCAGATTCGTGAAGGGCGACATCAGC |
| | ** ** ** * * * ** ** ** ** ** * ** ** ** ** ** ** * |
| wtPCCA | ACTAAATTTCTCTCCGATGTGTATCCTGATGGCTTCAAAGGACACATGCTAACCAAGAGT |
| synPCCA2 | ACTAAGTTCCTCTCCGACGTGTACCCAGACGGTTTTAAAGGGCACATGCTTACTAAGTCC |
| synPCCA3 | ACCAAGTTCCTGAGTGACGTATACCCCGACGGCTTTAAGGGGCATATGCTGACAAAGTCA |
| synPCCA6 | ACCAAGTTCCTGAGCGACGTGTACCCTGATGGTTTCAAGGGTCACATGCTGACTAAGTCC |
| synPCCA1 | ACCAAATTTCTGTCCGACGTGTACCCCGATGGCTTCAAGGGGCACATGCTGACAAAGTCT |
| synPCCA4 | ACCAAGTTTCTGTCCGACGTGTACCCCGACGGCTTCAAGGGACACATGCTGACCAAGAGC |
| synPCCA5 | ACCAAGTTCCTGAGCGACGTGTACCCCGACGGCTTCAAGGGCCACATGCTGACCAAGAGC |
| | ** ** ** ** ** ** ** ** ** ** ** ** ** ** ***** ** *** |
| wtPCCA | GAGAAGAACCAGTTATTGGCAATAGCATCATCATTGTTTGTGGCATTCCAGTTAAGAGCA |
| synPCCA2 | GAAAAGAATCAACTGTTGGCTATTGCGTCTTCCCTTTTTGTTGCTTTCCAACTGCGCGCG |
| synPCCA3 | GAGAAGAATCAACTCCTCGCAATAGCCAGTAGCCTGTTTGTTGCCTTCCAGCTGAGGGCT |
| synPCCA6 | GAGAAGAACCAGCTCCTCGCTATCGCGTCCTCCCTGTTTGTGGCGTTCCAGCTGAGGGCC |
| synPCCA1 | GAGAAAAATCAGCTGCTGCCTATCGCAAGTTCACTGTTCGTGGCATTTCAGCTGCGGGCC |
| synPCCA4 | GAGAAGAACCAGCTGCTCGCCATTGCCTCCAGCCTGTTTGTGGCCTTTCAGCTGAGAGCC |
| synPCCA5 | GAGAAGAACCAGCTGCTGCCCATCGCCAGCAGCCTGTTCGTGGCCTTCCAGCTGAGAGCC |
| | ** ** ** ** * * ** ** ** * ** ** ** ** ** * * ** |
| wtPCCA | CAACATTTTCAAGAAAATTCAAGAATGCCTGTTATTAAACCAGACATAGCCAACTGGGAG |
| synPCCA2 | CAGCATTTCCAGGAGAATAGCAGAATGCCCGTTATCAAACCTGATATTGCGAACTGGGAA |
| synPCCA3 | CAGCACTTCCAGGAGAATAGCAGAATGCCCGTTATCAAACCTGATATCGCGAATTGGGAA |
| synPCCA6 | CAGCACTTCCAAGAAAACTCAAGAATGCCGGTCATCAAGCCCGACATTGCCAATTGGGAA |
| synPCCA1 | CAGCATTTTCAGGAGAACAGTAGAATGCCCGTGATCAAGCCTGACATTGCAAATTGGGAA |
| synPCCA4 | CAGCACTTCCAAGAGAACAGCAGAATGCCCGTGATCAAGCCCGATATCGCCAACTGGGAG |
| synPCCA5 | CAGCACTTCCAGGAGAACAGCAGAATGCCCGTGATCAAGCCCGACATCGCCAACTGGGAG |
| | ** ** ** ** ** ** ******** ** ** ** ** ** ** ** ** ***** |
| wtPCCA | CTCTCAGTAAAATTGCATGATAAAGTTCATACCGTAGTAGCATCAAACAATGGGTCAGTG |
| synPCC72 | TTGTCAGTTAAGCTGCATGATAAGGTGCATACCGTAGTGGCTAGTAATAACGGAAGCGTT |
| synPCCA3 | TTGAGCGTGAAGCTGCACGATAAAGTTCATACTGTTGTGGCCTCAAACAATGGAAGCGTC |
| synPCC76 | CTGAGCGTGAAGCTGCACGACAAAGTGCACACCGTGGTGGCCAGCAACAACGGCTCCGTG |
| synPCCA1 | CTGAGTGTCAAGCTGCACGATAAAGTGCATACCGTGGTCGCTTCAAACAATGGCAGCGTG |
| synPCCA4 | CTGAGCGTGAAGCTGCACGATAAGGTGCACACAGTGGTGGCCAGCAACAACGGCTCCGTG |
| synPCCA5 | CTGAGCGTGAAGCTGCACGACAAGGTGCACACCGTGGTGGCCAGCAACAACGGCAGCGTG |
| | * ** ** **** ** ** ** ** ** ** ** ** ** ** ** ** |
| wtPCCA | TTCTCGGTGGAAGTTGATGGGTCGAAACTAAATGTGACCAGCACGTGGAACCTGGCTTCG |
| synPCCA2 | TTTTCCGTTGAAGTAGACGGCTCCAAGCTTAATGTGACGAGCACATGGAACCTTGCCTCT |
| synPCCA3 | TTTAGCGTGGAGGTCGATGGATCCAAACTGAACGTGACCAGTACCTGGAATTTGGCCAGT |
| synPCCA6 | TTCTCCGTGGAAGTGGATGGGTCAAAGCTGAACGTGACCAGCACCTGGAACCTGGCGTCC |
| synPCCA1 | TTCAGCGTCGAGGTGGACGGGTCTAAACTGAACGTGACCAGTACATGGAATCTGGCCTCA |
| synPCCA4 | TTCAGCGTGGAAGTGGACGGCAGCAAGCTGAACGTGACCTCCACCTGGAATCTGGCCTCT |
| synPCCA5 | TTCAGCGTGGAGGTGGACGGCAGCAAGCTGAACGTGACCAGCACCTGGAACCTGGCCAGC |
| | ** ** ** ** ** ** ** ** ** ***** ** ***** * ** |
| wtPCCA | CCCTTATTGTCTGTCAGCGTTGATGGCACTCAGAGGACTGTCCAGTGTCTTTCTCGAGAA |
| synPCCA2 | CCACTGCTTAGTGTGAGTGTGGACGGAACGCAGAGGACAGTTCAATGCCTGAGTCGGGAA |
| synPCCA3 | CCGCTGTTGTCTGTCTCCGTGGATGGAACGCAACGAACTGTGCAGTGTCTGTCTCGCGAA |
| synPCCA6 | CCGCTCCTGTCAGTGTCCGTGGACGGCACTCAGCGGACTGTGCAGTGTTTGTCCCGGGAA |
| synPCCA1 | CCACTGCTGTCAGTCAGCGTGGATGGCACACAGCGCACTGTGCAGTGCCTGAGCCGGGAG |
| synPCCA4 | CCACTGCTGTCCGTGTCTGTGGATGGCACCCAGAGAACCGTGCAGTGTCTGAGCAGAGAA |
| synPCCA5 | CCCCTGCTGAGCGTGAGCGTGGACGGCACCCAGAGAACCGTGCAGTGCCTGAGCAGAGAG |
| | ** * * ** ** ** ** ** ** * ** ** ** ** * * ** |
| wtPCCA | GCAGGTGGAAACATGAGCATTCAGTTTCTTGGTACAGTGTACAAGGTGAATATCTTAACC |
| synPCCA2 | GCGGGAGGTAACATGAGTATACAATTCCTCGGAACCGTCTATAAAGTTAACATTTTGACG |
| synPCCA3 | GCCGGAGGCAACATGAGCATTCAGTTTCTCGGGACTGTGTACAAAGTCAACATCCTGACC |
| synPCCA6 | GCCGGGGGCAATATGAGCATCCAGTTCCTCGGGACGGTGTACAAGGTCAACATCCTCACT |
| synPCCA1 | GCAGGAGGAAACATGAGTATTCAGTTTCTGGGGACTGTCTATAAGGTGAACATCCTGACC |
| synPCCA4 | GCAGGCGGCAATATGAGCATCCAGTTTCTGGGCACCGTGTACAAAGTGAACATCCTGACC |
| synPCCA5 | GCCGGCGGCAACATGAGCATCCAGTTCCTGGGCACCGTGTACAAGGTGAACATCCTGACC |
| | ** ** ** ** ***** ** ** ** ** ** ** ** ** ** ** ** ** * ** |
| wtPCCA | AGACTTGCCGCAGAATTGAACAAATTTATGCTGGAAAAAGTGACTGAGGACACAAGCAGT |
| synPCCA2 | AGATTGGCGGCTGAACTGAATAAGTTCATGCTCGAGAAAGTGACTGAGGACACTTCAAGC |
| synPCCA3 | CGACTGGCTGCCGAGCTGAACAAATTTATGCTTGAGAAAGTCACTGAGGATACGTCTAGC |
| synPCCA6 | CGGTTGGCCGCTGAACTCAACAAGTTCATGCTGGAAAAGGTCACCGAGGACACCTCCTCT |
| synPCCA1 | AGGCTGGCTGCAGAACTGAATAAGTTCATGCTGGAGAAAGTGACCGAAGACACAAGCTCC |
| synPCCA4 | AGACTGGCCGCTGAGCTGAACAAGTTCATGCTGGAAAAAGTGACCGAGGACACCAGCAGC |
| synPCCA5 | AGACTGGCCGCCGAGCTGAACAAGTTCATGCTGGAGAAGGTGACCGAGGACACCAGCAGC |
| | * * ** ** ** * ** ** ** ***** ** ** ** ** ** ** ** |
| wtPCCA | GTTCTGCGTTCCCCGATGCCCGGAGTGGTGGTGGCCGTCTCTGTCAAGCCTGGAGACGCG |
| synPCCA2 | GTACTGAGGAGCCCTATGCCGGGGGTTGTCGTAGCAGTGTCTGTTAAGCCAGGAGATGCG |
| synPCCA3 | GTOCTTOGGAGTCCTATGCCAGGGGTGGTGGTGGCCGTTTCAGTCAAACCAGGTGATGCC |
| synPCCA6 | GTGCTGOGGTCGCCCATGCCGGGAGTGGTCGTGGCCGTGTCCGTGAAGCCTGGCGATGCC |
| synPCCA1 | GTGCTGCGCTCACCAATGCCGAGAGTGGTCGTGGCCGTCAGCGTGAAGCCAGGGGATGCA |
| synPCCA4 | GTGCTGAGATCTCCTATGCCTGGTGTCGTGGTGGCCGTGTCAGTGAAACCTGGGGATGCT |
| synPCCA5 | GTGCTGAGAAGCCCCATGCCCGGCGTGGTGGTGGCCGTGAGCGTGAAGCCCGGCGACGCC |
| | ** ** * ** ***** ** ** ** ** ** ** ** ** ** ** ** ** |
| wtPCCA | GTAGCAGAAGGTCAAGAAATTTGTGTGATTGAAGCCATGAAAATGCAGAATAGTATGACA |
| synPCCA2 | GTGGCAGAAGGCCAAGAAATTTGCGTGATTGAGGCAATGAAAATGCAGAACTCAATGACC |
| synPCCA3 | GTAGCCGAAGGTCAGGAAATCTGCGTTATCGAGGCTATGAAGATGCAGAACAGCATGACA |
| synPCCA6 | GTGGCCGAAGGTCAAGAAATTTGCGTGATCGAGGCCATGAAGATGCAGAACTCGATGACG |
| synPCCA1 | GTGGCTGAGGGACAGGAGATTTGCGTGATTGAGGCTATGAAAATGCAGAACAGCATGACC |
| synPCCA4 | GTGGCCGAGGGCCAAGAGATCTGTGTGATCGAGGCCATGAAGATGCAGAACAGCATGACC |
| synPCCA5 | GTGGCCGAGGGCCAGGAGATCTGCGTGATCGAGGCCATGAAGATGCAGAACAGCATGACC |
| | ** ** ** ** ** ** ** ** ** ** ** ** ***** ******** ***** |
| wtPCCA | GCTGGGAAAACTGGCACGGTGAAATCTGTGCACTGTCAAGCTGGAGACACAGTTGGAGAA |
| synPCCA2 | GCCGGAAAAACGGGCACGGTCAAATCTGTGCATTGTCAGGCAGGCGACACAGTCGGCGAG |
| synPCCA3 | GCCGGGAAAACCGGAACAGTGAAGTCAGTTCATTGCCAGGCTGGGGACACAGTCGGCGAG |
| synPCCA6 | GCCGGAAAGACCGGCACCGTCAAAAGCGTGCACTGCCAGGCCGGCGATACCGTGGGAGAG |
| synPCCA1 | GCAGGAAAGACTGGCACCGTGAAAAGCGTGCATTGTCAGGCTGGGGATACTGTCGGGGAA |
| synPCCA4 | GCCGGCAAGACCGGCACAGTGAAGTCTGTGCATTGTCAGGCCGGCGATACAGTCGGAGAA |
| synPCCA5 | GCCGGCAAGACCGGCACCGTGAAGAGCGTGCACTGCCAGGCCGGCGACACCGTGGGCGAG |
| | ** ** ** ** ** ** ** ** ** ** ** ** ** ** ** ** ** ** ** |
| wtPCCA | GGGGATCTGCTCGTGGAGCTGGAATGA |
| synPCCA2 | GGTGATCTCCTGGTAGAGTTGGAATGA |
| synPCCA3 | GGCGATTTGCTGGTGGAACTGGAATGA |
| synPCCA6 | GGCGATCTGCTCGTGGAACTCGAATGA |
| synPCCA1 | GGGGATCTGCTGGTGGAACTGGAGTGA |
| synPCCA4 | GGCGATCTGCTGGTGGAACTGGAATGA |
| synPCCA5 | GGCGACCTGCTGGTGGAGCTGGAGTGA |
| | ** ** * ** ** ** * ** *** |

In another aspect, SEQ ID NOs:2-7 encode a PCC alpha subunit that has 100% identity with the naturally occurring human PCC alpha subunit protein, or that has at least 90% amino acid identity to the naturally occurring human PCC alpha subunit protein. In a preferred embodiment, the polynucleotide encodes a PCC alpha subunit protein that has at least 95% amino acid identity to naturally occurring human PCC alpha subunit protein.

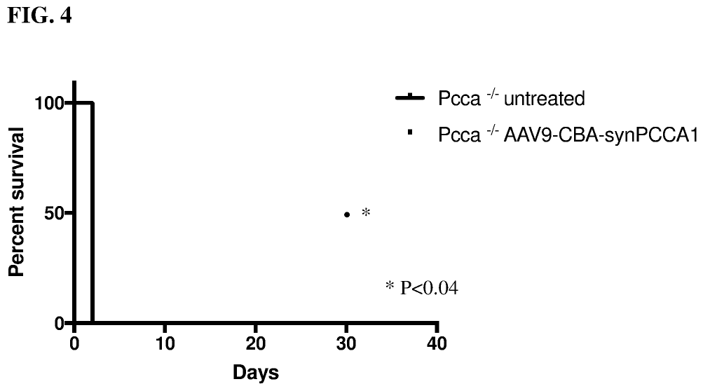

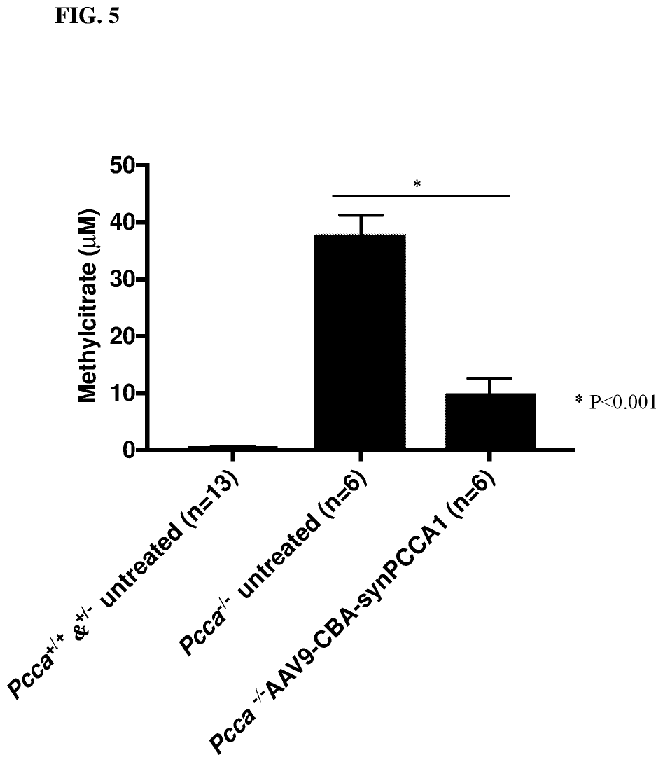

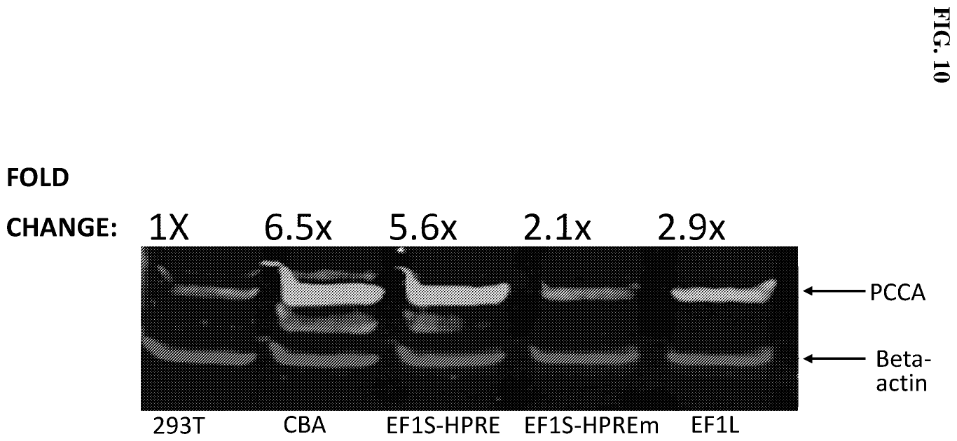

In one embodiment, a polypeptide according to the invention retains at least 90% of the naturally occurring human PCC protein function, i.e., the capacity to catalyze the carboxylation of propionyl-CoA to D-methylmalonyl-CoA. In another embodiment, the encoded PCC protein retains at least 95% of the naturally occurring human PCC protein function. This protein function can be measured, for example, via the efficacy to rescue a neonatal lethal phenotype in Pcca knock-out mice (FIGS. 4, 10), the lowering of circulating metabolites including 2-methylcitrate in a disease model of PA (FIG. 5).

In some embodiments, the synthetic polynucleotide exhibits improved expression relative to the expression of naturally occurring human propionyl-CoA carboxylase alpha polynucleotide sequence. The improved expression is due to the polynucleotide comprising codons that have been optimized relative to the naturally occurring human propionyl-CoA carboxylase alpha polynucleotide sequence. In one aspect, the synthetic polynucleotide has at least about 25%, at least about 30%, at least about 35%, at least about 40%, at least about 45%, at least about 50%, at least about 55%, at least about 60%, at least about 65%, at least about 70%, at least about 75%, at least about 80% of less commonly used codons replaced with more commonly used codons. In additional embodiments, the polynucleotide has at least 85%, 90%, or 95% replacement of less commonly used codons with more commonly used codons, and demonstrate equivalent or enhanced expression of PCCA as compared to SEQ ID NO:1.

In some embodiments, the synthetic polynucleotide sequences of the invention preferably encode a polypeptide that retains at least about 80% of the enhanced PCC expression (as demonstrated by expression of the polynucleotide of SEQ ID NO:1 in an appropriate host.) In additional embodiments, the polypeptide retains at least 85%, 90%, or 95% or 100% of the enhanced expression observed with the polynucleotides of SEQ ID NOs: 2-7.

In designing the synPCCA of the present invention, the following considerations were balanced. For example, the fewer changes that are made to the nucleotide sequence of SEQ ID NO:1, decreases the potential of altering the secondary structure of the sequence, which can have a significant impact on gene expression. The introduction of undesirable restriction sites is also reduced, facilitating the subcloning of PCCA into the plasmid expression vector. However, a greater number of changes to the nucleotide sequence of SEQ ID NO:1 allows for more convenient identification of the translated and expressed message, e.g. mRNA, in vivo. Additionally, greater number of changes to the nucleotide sequence of SEQ ID NO:1 provides for increased likelihood of greater expression. These considerations were balanced when arriving at SEQ ID NOs: 2-7. The polynucleotide sequences encoding synPCCA allow for increased expression of the synPCCA gene relative to naturally occurring human PCCA sequences. They are also engineered to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. Because the sequences are novel, they facilitate detection using nucleic acid-based assays.

PCCA has a total of 728 amino acids and synPCCA contains 728 codons corresponding to said amino acids. In SEQ ID NOs: 2-7, codons are changed from that of the natural human PCCA, however, as described, SEQ ID NOs: 2-7, despite changes from SEQ ID NO:1, codes for the amino acid sequence SEQ ID NO:8 for PCCA. Codons for SEQ ID NOs: 2-7 are changed, in accordance with the equivalent amino acid positions of SEQ ID NO:8, as seen in Table 2. In this embodiment, the amino acid sequence for natural human PCCA has been retained.

It can be appreciated that partial reversion of the designed synPCCA to codons that are found in PCCA can be expected to result in nucleic acid sequences that, when incorporated into appropriate vectors, can also exhibit the desirable properties of SEQ ID NOs: 2-7, for example, such partial reversion or hybrid variants can have equivalent expression of PCCA from a vector inserted into an appropriate host, as SEQ ID NOs: 2-7. For example, the invention includes nucleic acids in which at least about 1 altered codon, at least about 2 altered codons, at least about 3, altered codons, at least about 4 altered codons, at least about 5 altered codons, at least about 6 altered codons, at least about 7 altered codons, at least about 8 altered codons, at least about 9 altered codons, at least about 10 altered codons, at least about 11 altered codons, at least about 12 altered codons, at least about 13 altered codons, at least about 14 altered codons, at least about 15 altered codons, at least about 16 altered codons, at least about 17 altered codons, at least about 18 altered codons, at least about 20 altered codons, at least about 25 altered codons, at least about 30 altered codons, at least about 35 altered codons, at least about 40 altered codons, at least about 50 altered codons, at least about 55 altered codons, at least about 60 altered codons, at least about 65 altered codons, at least about 70 altered codons, at least about 75 altered codons, at least about 80 altered codons, at least about 85 altered codons, at least about 90 altered codons, at least about 95 altered codons, at least about 100 altered codons, at least about 110 altered codons, at least about 120 altered codons, at least about 130 altered codons, at least about 130 altered codons, at least about 140 altered codons, at least about 150 altered codons, at least about 160 altered codons, at least about 170 altered codons, at least about 180 altered codons, at least about 190 altered codons, at least about 200 altered codons, at least about 220 altered codons, at least about 240 altered codons, at least about 260 altered codons, at least about 280 altered codons, at least about 300 altered codons, at least about 320 altered codons, at least about 340 altered codons, at least about 360 altered codons, at least about 380 altered codons, at least about 400 altered codons, at least about 420 altered codons, at least about 440 altered codons, at least about 460 altered codons, or at least about 480 of the altered codon positions in SEQ ID NOs: 2-7 are reverted to native codons according to SEQ ID NO:1, and having equivalent expression to SEQ ID NO:1. Alternately, at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% of the altered codon positions in SEQ ID NOs:2-7 are reverted to native sequence according to SEQ ID NO:1, and having equivalent expression to SEQ ID NOs: 2-7.

In some embodiments, polynucleotides of the present invention do not share 100% identity with SEQ ID NO:1. In other words, in some embodiments, polynucleotides having 100% identity with SEQ ID NO:1 are excluded from the embodiments of the present invention.

The synthetic polynucleotide can be composed of DNA and/or RNA or a modified nucleic acid, such as a peptide nucleic acid, and could be conjugated for improved biological properties.

### Therapy

In another aspect, the invention comprises a method of treating a disease or condition mediated by propionyl-CoA carboxylase. The disease or condition can, in one embodiment, be propionic acidemia (PA). This method comprises administering to a subject in need thereof a synthetic propionyl-CoA carboxylase polynucleotide construct comprising the synthetic polynucleotides (synPCCA) described herein. The PCC enzyme is processed after transcription, translation, and translocation into the mitochondrial inner space.

Enzyme replacement therapy consists of administration of the functional enzyme (propionyl-CoA carboxylase) to a subject in a manner so that the enzyme administered will catalyze the reactions in the body that the subject's own defective or deleted enzyme cannot. In enzyme therapy, the defective enzyme can be replaced in vivo or repaired in vitro using the synthetic polynucleotide according to the invention. The functional enzyme molecule can be isolated or produced in vitro, for example. Methods for producing recombinant enzymes in vitro are known in the art. In vitro enzyme expression systems include, without limitation, cell-based systems (bacterial (for example, Escherichia coli, Corynebacterium, Pseudomonas fluorescens), yeast (for example, Saccharomyces cerevisiae, Pichia Pastoris), insect cell (for example, Baculovirus-infected insect cells, non-lytic insect cell expression), and eukaryotic systems (for example, Leishmania)) and cell-free systems (using purified RNA polymerase, ribosomes, tRNA, ribonucleotides). Viral in vitro expression systems are likewise known in the art. The enzyme isolated or produced according to the above-iterated methods exhibits, in specific embodiments, 80%, 85%, 90%, 95%, 98%, 99%, or 100% homology to the naturally occurring (for example, human) propionyl-CoA carboxylase.

Gene therapy can involve in vivo gene therapy (direct introduction of the genetic material into the cell or body) or ex vivo gene transfer, which usually involves genetically altering cells prior to administration. In one aspect, genome editing, or genome editing with engineered nucleases (GEEN) may be performed with the synPCCA nucleotides of the present invention allowing synPCCA DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the nucleotides of the present invention including synPCCA, in combination with a CASP/CRISPR, ZFN, or TALEN can be used to engineer correction at the locus in a patient's cell either in vivo or ex vivo, then, in one embodiment, use that corrected cell, such as a fibroblast or lymphoblast, to create an iPS or other stem cell for use in cellular therapy.

In another embodiment, the synPCCA nucleotides of the present invention can be used in combination with a non-integrating vector or as naked DNA, and configured to contain terminal repeat sequences for a transposon recognition by a transposase such as piggyBac. The use of hybrid AAV and adenoviral vectors that combine the transient or regulated expression of a transposase like piggyBac may be performed to enable permanent correction by cut and paste transposition. Alternatively, the transposase mRNA, encapsulated as lipid-nanoparticle, might be used to deliver piggBac transposase.

### Administration/Delivery and Dosage Forms

Routes of delivery of a synthetic propionyl-CoA carboxylase (PCCA) polynucleotide according to the invention may include, without limitation, injection (systemic or at target site), for example, intradermal, subcutaneous, intravenous, intraperitoneal, intraocular, subretinal, renal artery, hepatic vein, intramuscular injection; physical, including ultrasound (-mediated transfection), electric field-induced molecular vibration, electroporation, transfection using laser irradiation, photochemical transfection, gene gun (particle bombardment); parenteral and oral (including inhalation aerosols and the like). Related methods include using genetically modified cells, antisense therapy, and RNA interference.

Vehicles for delivery of a synthetic propionyl-CoA carboxylase polynucleotide (synPCCA) according to the invention may include, without limitation, viral vectors (for example, AAV, integrating AAV vectors, adenovirus, baculovirus, retrovirus, lentivirus, foamy virus, herpes virus, Moloney murine leukemia virus, Vaccinia virus, and hepatitis virus) and non-viral vectors (for example, naked DNA, mini-circles, liposomes, ligand-polylysine-DNA complexes, nanoparticles, including mRNA containing lipid nanoparticles, cationic polymers, including polycationic polymers such as dendrimers, synthetic peptide complexes, artificial chromosomes, and polydispersed polymers). Thus, dosage forms contemplated include injectables, aerosolized particles, capsules, and other oral dosage forms.

In certain embodiments, the vector used for gene therapy comprises an expression cassette. The expression cassette may, for example, consist of a promoter, the synthetic polynucleotide, and a polyadenylation signal. Viral promoters include, for example, the ubiquitous cytomegalovirus immediate early (CMV-IE) promoter, the chicken beta-actin (CBA) promoter, the simian virus 40 (SV40) promoter, the Rous sarcoma virus long terminal repeat (RSV-LTR) promoter, the Moloney murine leukemia virus (MoMLV) LTR promoter, and other retroviral LTR promoters. The promoters may vary with the type of viral vector used and are well-known in the art.

In one specific embodiment, synPCCA could be placed under the transcriptional control of a ubiquitous or tissue-specific promoter, with a 5′ intron, 5′ intron translational enhancer element, and flanked by an mRNA stability element, such as the woodchuck or hepatitis post-transcriptional regulatory element, and polyadenylation signal. The use of a tissue-specific promoter can restrict unwanted transgene expression, as well as facilitate persistent transgene expression. The therapeutic transgene could then be delivered as coated or naked DNA into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

In another embodiment, the same synPCCA expression construct could be packaged into a viral vector, such as an adenoviral vector, retroviral vector, lentiviral vector, or adeno-associated viral vector, and delivered by various means into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

Tissue-specific promoters include, without limitation, Apo A-I, ApoE, hAAT, transthyretin, liver-enriched activator, albumin, TBG, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, CaMK2, and myelin basic protein promoters (neuronal), and ankyrin, α-spectrin, globin, HLA-DRα, CD4, glucose 6-phosphatase, and dectin-2 promoters (erythroid).

Regulable promoters (for example, ligand-inducible or stimulus-inducible promoters) and optogenetic promoters are also contemplated for expression constructs according to the invention.

In yet another embodiment, synPCCA could be used in ex vivo applications via packaging into a retro or lentiviral vector to create an integrating vector that could be used to permanently correct any cell type from a patient with PCC deficiency. The synPCCA-transduced and corrected cells could then be used as a cellular therapy. Examples might include CD34+ stem cells, primary hepatocytes, or fibroblasts derived from patients with PCC deficiency. Fibroblasts could be reprogrammed to other cell types using iPS methods well known to practitioners of the art. In yet another embodiment, synPCCA could be recombined using genomic engineering techniques that are well known to practitioners of the art, such as ZFNs and TALENS, into the PCCA locus, a genomic safe harbor site, such as AAVS1, or into another advantageous location, such as into rDNA, the albumin locus, GAPDH, or a suitable expressed pseudogene. In yet another embodiment, synPCCA could be delivered using a hybrid AAV-piggyBac transposon system as is well known to practitioners of the art (see PMID: 31099022), and references therein:
Prevention of Cholestatic Liver Disease and Reduced Tumorigenicity in a Murine Model of PFIC Type 3 Using Hybrid AAV-piggyBac Gene Therapy. Siew S M, Cunningham S C, Zhu E, Tay S S, Venuti E, Bolitho C, Alexander I E. Hepatology. 2019 December; 70(6):2047-2061. PMID: 31099022.)

A composition (pharmaceutical composition) for treating an individual by gene therapy may comprise a therapeutically effective amount of a vector comprising the synPCCA transgenes or a viral particle produced by or obtained from same. The pharmaceutical composition may be for human or animal usage. Typically, a physician will determine the actual dosage which will be most suitable for an individual subject, and it will vary with the age, weight, and response of the particular individual.

The composition may, in specific embodiments, comprise a pharmaceutically acceptable carrier, diluent, excipient, or adjuvant. Such materials should be non-toxic and should not interfere with the efficacy of the transgene. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences [Mack Pub. Co., 18th Edition, Easton, Pa. (1990)]. The choice of pharmaceutical carrier, excipient, or diluent can be selected with regard to the intended route of administration and standard pharmaceutical practice. The pharmaceutical compositions may comprise as, or in addition to, the carrier, excipient, or diluent any suitable binder(s), lubricant(s), suspending agent(s), coating agent(s), solubilizing agent(s), and other carrier agents that may aid or increase the viral entry into the target site (such as for example a lipid delivery system). For oral administration, excipients such as starch or lactose may be used. Flavoring or coloring agents may be included, as well. For parenteral administration, a sterile aqueous solution may be used, optionally containing other substances, such as salts or monosaccharides to make the solution isotonic with blood.

A composition according to the invention may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, or drugs (e.g., antibiotics).

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. Additional dosage forms contemplated include: in the form of a suppository or pessary; in the form of a lotion, solution, cream, ointment or dusting powder; by use of a skin patch; in capsules or ovules; in the form of elixirs, solutions, or suspensions; in the form of tablets or lozenges.

### Examples

Cell culture studies: Six synthetic codon-optimized human propionyl-CoA carboxylase subunit alpha genes (synPCCA1-6) were engineered using an iterative approach, wherein the naturally occurring PCCA cDNA (NCBI Reference Sequence: NM_000282.4) was optimized codon by codon to create (synPCCA1-6) (SEQ ID NOs: 2-7), using a variety of codon optimization methods, one of which incorporated critical factors involved in protein expression, such as codon adaptability, mRNA structure, and various cis-elements in transcription and translation. The resulting sequences were manually inspected and subject to expert adjustment. The synPCCA alleles displayed maximal divergence from the PCCA cDNA at the nucleotide level yet retained optimally utilized codons at each position.

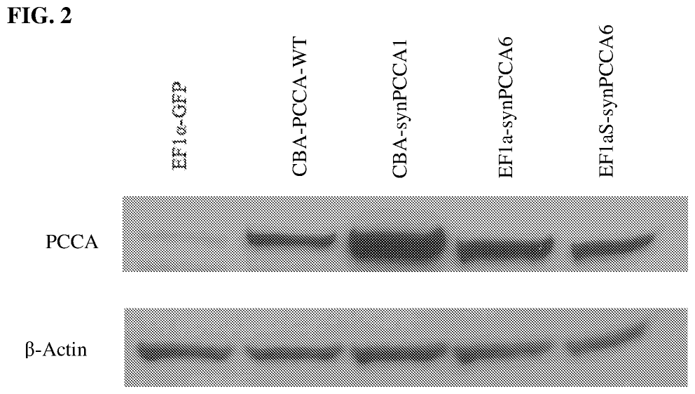

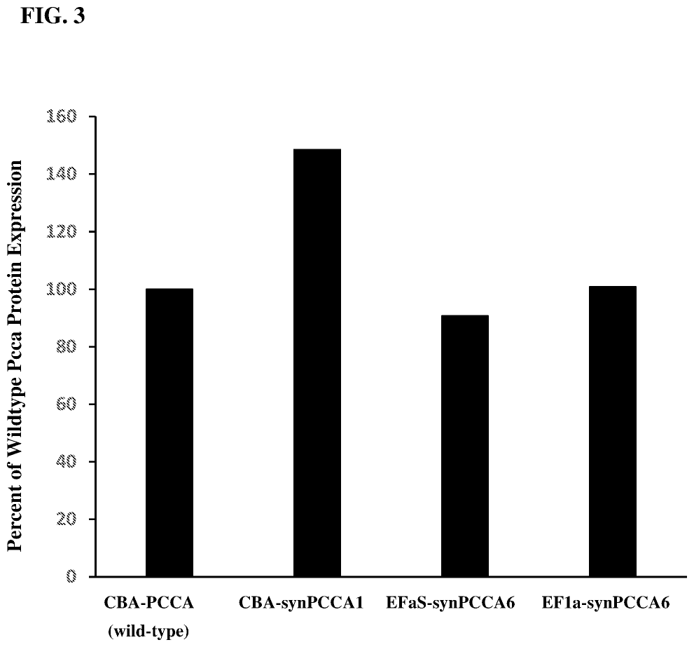

To improve the expression of propionyl-CoA carboxylase and create a vector that could express the human PCCA gene in a more efficient fashion, synPCCA1 was cloned using restriction endonuclease excision and DNA ligation into an expression vector under the control of the strong chicken β-actin promoter (CBA) (Chandler, et al. 2010 Mol Ther 18:11-6) or the active but not as potent elongation factor 1 alpha promoter (EF1a). The constructs expressing either PCCA or synPCCA1 with the CBA or synPCCA6 with EF1α long or short promoters were then transfected into 293FT cells using Lipofectamine™ (Life Technologies). Cloning and transfection methods are well understood by practitioners of the art (Sambrook, Fritsch, Maniatis. Molecular Cloning: A Laboratory Manual). After 48 hours, cellular protein was extracted from the transfected cells and evaluated for propionyl-CoA carboxylase protein expression using Western analysis (Chandler, et al. 2010 Mol Ther 18:11-6). The results show that synPCCA1 is expresses 140% the level of the wild type human PCCA1 gene (FIGS. 2 and 3) and also that synPCCA6 is transcribed and translated as well as or more efficiently than PCCA (FIGS. 2 and 3). Of interest, synPCCA6 expresses PCCA at levels close to the wild-type control CBA-PCCA even when expressed under the less potent EF1a promoters (FIGS. 2 and 3).

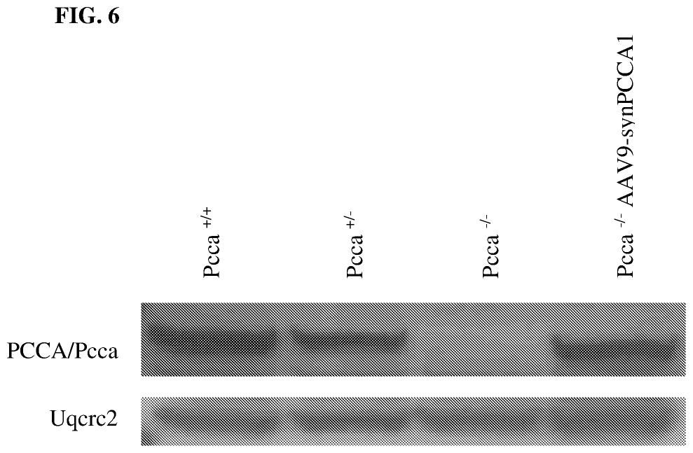

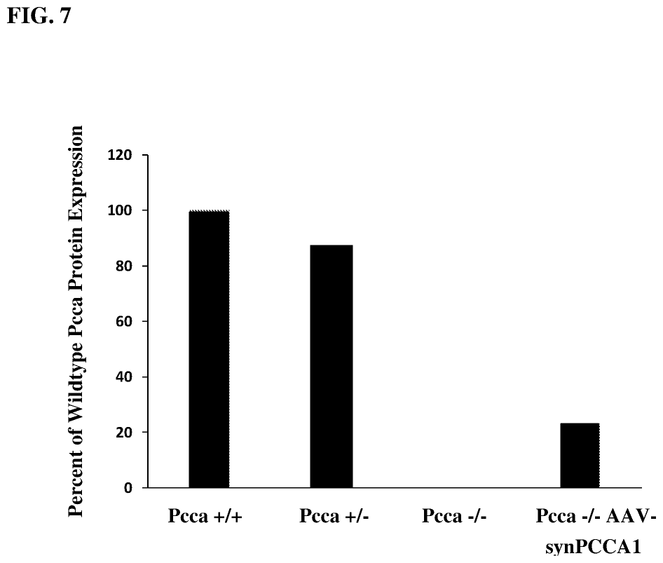

AAV9 gene therapy in propionyl-CoA carboxylase Knock-out (Pcca^{-/-}) Mice. The promising expression data from both constructs led to the production of AAV9-CBA-synPCCA1 which was delivered to neonatal Pcca^{-/-} mice. As presented in FIG. 4, 50% of the Pcca^{-/-} mice that received the AAV lived to 30 days, and further had a wild type appearance, as compared to the untreated Pcca^{-/-} mice which had 100% mortality in early life. The surviving mice were sacrificed at 30 days for metabolic studies and to examine hepatic transgene expression. A substantial reduction in the disease related metabolite methylcitrate accompanied the rescue as seen in FIG. 5. Finally, a Western blot using murine livers, from wild-type mice (Pcca^{+/+} and Pcca^{+/-}), an untreated Pcca^{-/-} mouse, and a Pcca^{-/-} mouse treated with 3×10¹¹ VC of AAV9-CBA-synPCCA1, was performed. As seen in FIG. 6 and FIG. 7, the treated Pcca^{-/-} mouse displayed robust hepatic PCCA expression whereas the untreated Pcca^{-/-} mouse showed no hepatic murine Pcca expression. It should be noted that the antibody used for Western blotting can detect both human (PCCA) and murine (Pcca) enzymes.

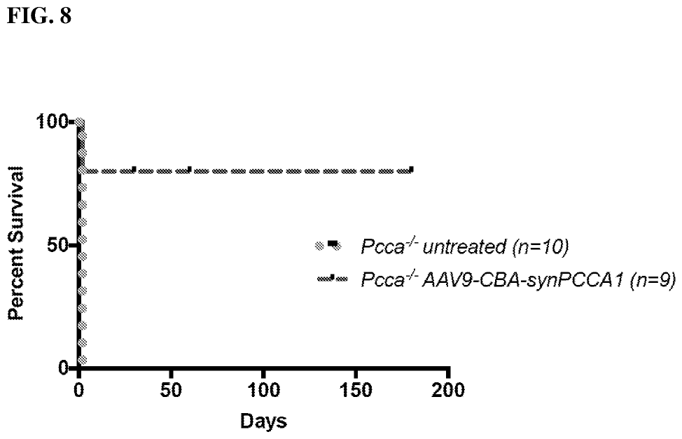

In a similar study, long term survival of neonatal AAV9-CBA-synPCCA1 treated Pcca^{-/-} mice was performed. Untreated Pcca^{-/-} (n=10) mice served as a control and were compared to Pcca^{-/-} mice (n=9) treated with 3×10¹¹ VC of AAV-CBA-synPCCA1 delivered by intrahepatic injection at birth. As can be seen in FIG. 8, treated Pcca^{-/-} mice display a significant increase in survival to >150 days. The AAV9-CBA-synPCCA1 treated Pcca^{-/-} mice mice remain alive at the time of this application.

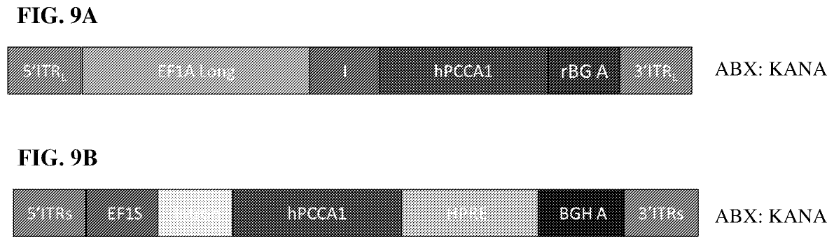

Next, a series of vectors designed to express synPCCA1 from the long elongation factor 1 alpha promoter EF1 or short elongation factor 1 alpha promoter (EF1AS) in combination with a 3′ the hepatitis B post translation response element (HPRE). FIG. 9A shows a vector comprised of 145 base pair AAV2 inverted terminal repeats (5′ITRL and 3′ ITRL), the long elongation factor 1 alpha promoter (EF1AL), an intron (I), the synPCCA1 gene, the rabbit beta-globin polyadenylation signal (rBGA). The production plasmid expresses the kanamycin resistance gene. FIG. 9B shows a vector comprised of 130 base pair AAV2 inverted terminal repeats (5′ITRS and 3′ ITRS), the short elongation factor 1 alpha promoter (EF1AS), an intron (I), synPCCA1 gene, the hepatitis B post translation response element (HPRE), and the bovine growth hormone polyadenylation signal (BGHA). The production plasmid expresses the kanamycin resistance gene.

The vectors were studied for expression in human cells. FIG. 10 presents a western blot showing PCCA protein expression in 293 cells, which are human transformed kidney cells, after transfection with transfected with AAV backbones expressing synPCCA1 under the control of various promoter/enhancer combinations. Cloning and transfection methods are well understood by practitioners of the art (Sambrook, Fritsch, Maniatis. Molecular Cloning: A Laboratory Manual). After 48 hours, cellular protein was extracted from the transfected cells and evaluated for propionyl-CoA carboxylase protein expression using Western analysis (Chandler, et al. 2010 Mol Ther 18:11-6). PCCA=propionyl-CoA carboxylase alpha subunit, CBA=chicken beta actin, EF1a=elongation factor 1 alpha, EF1aS=elongation factor 1 alpha short. HPRE—hepatitis B post translation response element. HPREm—hepatitis B post translation response element, mutant. Beta-actin is the loading control. Compared to the untransfected cells (lane 1), the AAV plasmids expressed variably, with the CBA cassette (lane 2) showing 6.5× expression of the untransfected cells, the EF1S-HPRE cassette showing 5.6× expression of the untransfected cells (lane 3), the EF1S-HPREm cassette showing 2.1× expression of the untransfected cells (lane 4), and the EF1L cassette showing 2.9× the expression of untransfected cells. The results reveal that the EF1S-HPRE and EF1L cassettes substantially overexpress PCC.

Next, AAV9 vectors were prepared using methods well known to practitioners (Chandler, et al. 2010 Mol Ther 18:11-6) and used to treat Pcca^{-/-} mice. FIG. 11 depicts survival in untreated Pcca^{-/-} (n=12) mice compared to Pcca^{-/-} mice (n=9) treated with 1×10¹¹ VC of AAV9-EF1aL-synPCCA1 (n=18), 1×10¹¹ VC of AAV9-EF1aS-synPCCA1-HPRE (n=15), or 4×10¹¹ VC of AAV9-EF1aS-synPCCA1-HPRE (n=5) delivered by retroorbital injection at birth. The treated Pcca^{-/-} mice display a significant increase in survival, with many mice remaining alive at the time of this application.

Animal studies were reviewed and approved by the National Human Genome Research Institute Animal User Committee. Hepatic injections were performed on non-anesthetized neonatal mice, typically within several hours after birth. Viral particles were diluted to a total volume of 20 microliters with phosphate-buffered saline immediately before injection and were delivered into the liver parenchyma using a 32-gauge needle and transdermal approach, as previously described.

Treatment with synPCCA polynucleotide delivered using an AAV (adeno-associated virus) rescued the Pcca^{-/-} mice from neonatal lethality (FIGS. 4,8,11), improved their growth, and lowered the levels of plasma methylcitrate in the blood (FIG. 5). This establishes the preclinical efficacy of synPCCA as a treatment for PA in vivo, including in other animal models, as well as in humans.

1. A synthetic propionyl-CoA carboxylase subunit a (PCCA) polynucleotide (synPCCA) selected from the group consisting of:
a) a polynucleotide comprising the nucleic acid sequence of any one of SEQ ID NOs: 2-7;
b) a polynucleotide comprising a polynucleotide having a nucleic acid sequence with at least about 80% identity to the nucleic acid sequence of any one of SEQ ID NOs: 2-7 and encoding a polypeptide according to SEQ ID NO:8, and having equivalent expression in a host to either expression of any one of SEQ ID NOs: 2-7 or SEQ ID NO:1 expression, wherein the polynucleotide does not have the nucleic acid sequence of SEQ ID NO:1.

2. The synthetic polynucleotide of claim 1, wherein:
(a) the polynucleotide has at least about 90% identity to the nucleic acid sequence of any one of SEQ ID NOs: 2-7;
(b) the polynucleotide has at least about 95% identity to the nucleic acid sequence of any one of SEQ ID NOs: 2-7;
(c) the synthetic PCCA gene is flanked by a 5′ untranslated region (5′UTR) that includes a strong Kozak translational initiation signal;
(d) the polynucleotide further comprises the wood chuck post-translational response element (SEQ ID: 31) or the hepatitis post-translational response element (SEQ ID: 32);
(e) the synthetic PCCA gene is configured to integrate into the genome after delivery using a lentiviral vector;
(f) the sequence selected from the group consisting of SEQ ID NOs: 2-7 exhibits increased expression in an appropriate host relative to the expression of SEQ ID NO:1 in an appropriate host; or
(g) the nucleic acid sequence has at least about 70% of less commonly used codons replaced with more commonly used codons.

3-4. (canceled)

5. The synthetic polynucleotide of claim 2, wherein the synthetic polynucleotide having increased expression comprises a nucleic acid sequence comprising codons that have been optimized relative to the naturally occurring human propionyl-CoA carboxylase subunit a polynucleotide sequence (SEQ ID NO:1).

6. (canceled)

7. A recombinant expression vector comprising the synthetic polynucleotide of claim 1.

8. The recombinant vector of claim 7, wherein the vector is a recombinant adeno-associated virus (rAAV), said rAAV comprising an AAV capsid, and a vector genome packaged therein, said vector genome comprising:
(a) a 5′-inverted terminal repeat sequence (5′-ITR) sequence;
(b) a promoter sequence;
(c) a partial fragment or complete coding sequence for PCCA; and
(d) a 3′-inverted terminal repeat sequence (3′-ITR) sequence.

9. The rAAV according to claim 8, wherein:
(a) the vector is comprised of the structure in FIG. 9A;
(b) the AAV capsid is from an AAV of serotype 8 or serotype 9;
(c) the vector further comprises terminal repeat sequences (SEQ ID: 33-34) from the piggyBac transposon, located after the 5′AAV ITR and before the 3′ AAV ITR; or
(d) the promoter is a tissue-specific promoter;
optionally wherein the tissue specific promoter promotor is selected from the group consisting of Apo A-I, ApoE, hAAT, transthyretin, liver-enriched activator, albumin, TBG, PEPCK, and RNAPII promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).

10. The rAAV according to claim 7, wherein:
(a) the AAV capsid is from an AAV of serotype 1, 2, 3, 4, 5, 6, 7, 8, 9, 10, 11, 12, 13, rh 10, hu37 or Anc, and mutants thereof or
(b) wherein the rAAV further comprises terminal repeat sequences recognized by piggyBac transposase.

11-13. (canceled)

14. The rAAV according to claim 8, wherein:
(a) the promoter is selected from the group consisting of chicken-beta actin promoter (SEQ ID NO: 9), the elongation factor 1 alpha long promoter (EF1AL) (SEQ ID NO:10), the elongation factor 1 alpha short promoter with a 3′ hepatitis B post translation response element (HPRE) (SEQ ID NO:11), and the short elongation factor 1 alpha promoter with a mutant 3′ hepatitis B post translation response element (HPRE) (SEQ ID NO:12);
(b) the promoter is selected from the group consisting of liver specific enhancer and promoter, such as the long (SEQ ID NO:14), or short variants (SEQ ID NO:13) of the apolipoprotein E enhancer, and wherein the promoter is operably linked to the long (SEQ ID NO:16) or short variants of the human alpha 1 antitrypsin promoter (SEQ ID NO:15), and optionally at least one intron selected from the group consisting of a chimeric intron (SEQ ID NO:17), modified B-globin intron (SEQ ID NO: 18), and a synthetic intron (SEQ ID NO:19); or
(c) the promoter is selected from the group consisting of a liver specific enhancer and promoters of a long (SEQ ID NO:14), or short variant (SEQ ID NO:13) of the apolipoprotein E enhancer, the enhanced human alpha 1 antitrypsin promoter (SEQ ID:36), and the enhanced TBG promoter (SEQ ID:35), wherein the promoter is operably linked to the long (SEQ ID NO:16) or short variants of the human alpha 1 antitrypsin promoter (SEQ ID NO:15) and followed by either a chimeric intron (SEQ ID NO:17), modified B-globin intron (SEQ ID NO: 18), or a synthetic intron (SEQ ID NO:19).

15-16. (canceled)

17. The rAAV according to claim 14, wherein:
(a) the apolipoprotein E enhancer, and the human alpha 1 antitrypsin promoter are operably linked to form a short (SEQ ID NO: 20) or long liver specific enhancer-promoter units (SEQ ID NO: 21) and placed 5′ to an intron selected from SEQ ID NO:17-19;
(b) the liver specific enhancer is derived from sequences upstream of the alpha-1-microglobulin/bikunin precursor (SEQ ID:23 and SEQ ID:24), and operably linked to the human thyroxine-binding globulin promoter (TBG) (SEQ ID:25); or
(c) the liver specific enhancer and human thyroxine-binding globulin promoter is SEQ ID:26.

18. The rAAV according to claim 17, wherein:
(a) the intron is the modified β-globin intron (SEQ ID NO: 18); or
(b) the intron comprises SEQ ID:22.

19-22. (canceled)

23. The synthetic polynucleotide of claim 2, wherein:
(a) the synthetic polynucleotide further comprises an internal ribosome entry site (IRES) (SEQ ID: 27) instead of, or in addition to, a UTR; or
(b) the UTR comprises sequences selected from the group consisting of human albumin (SEQ ID: 28), SERPINA 1 (SEQ ID: 29), and SERPINA 3 (SEQ ID: 30);
optionally wherein the synthetic polynucleotide further comprises:
(i) at least one translation enhancer element (TEE), optionally wherein (i) the TEE is located between the promoter and the start codon or (ii) the 5′UTR comprises a TEE;
(ii) a donor cassette that targets the stop codon of human albumin, which yields, after homologous recombination synPCCA1 fused via a P2 peptide to the carboxy terminus of albumin; or
(iii) an integrating AAV vector, from 5′ITR to 3′ITR, that uses homologous recombination to insert synPCCA1 into end of human Albumin, having a safe harbor for gene editing, is SEQ ID:37.

24-28. (canceled)

29. The synthetic polynucleotide of claim 1, further comprising:
(a) a polyadenylation signal, optionally wherein the polyadenylation signal is a rabbit beta globin gene or the bovine growth hormone gene;
(b) a donor cassette that targets the stop codon of human albumin, which yields, after homologous recombination synPCCA1 fused via a P2 peptide to the carboxy terminus of albumin;
(c) an integrating AAV vector, from 5′ITR to 3′ITR, that uses homologous recombination to insert synPCCA1 into end of human Albumin, having a safe harbor for gene editing, is SEQ ID:37; or
(d) an integrating AAV vector, from 5′ITR to 3′ITR, that uses homologous recombination to insert synPCCA1 into 5′ end of human Albumin is SEQ ID:38.

30-35. (canceled)

36. The synthetic polynucleotide of claim 2, wherein:
(a) the lentiviral vector further comprises an enhanced human alpha 1 antitrypsin enhancer, and the promoter is SEQ ID: 39; or
(b) the lentiviral vector further comprises the elongation factor 1 long promoter is SEQ ID:40.

37-39. (canceled)

40. The expression vector of claim 7, wherein:
(a) the expression vector is AAV2/9-CBA-synPCCA1;
(b) the expression vector is AAV2/9-EF1L-synPCCA1;
(c) the expression vector is AAV2/9-EF1S-HPRE synPCCA1; or
(d) the expression vector is AAV2/9-EF1S-mHPRE synPCCA1.

41-43. (canceled)

44. A composition comprising the synthetic polynucleotide of claim 1 or a recombinant expression vector comprising the polynucleotide and a pharmaceutically acceptable carrier, optionally wherein the composition further comprises a hybrid AAV-piggyBac transposon system.

45-46. (canceled)

47. A method of treating a disease or condition mediated by propionyl-CoA carboxylase, comprising administering to a subject in need thereof a therapeutic amount of the synthetic polynucleotide of claim 1.

48. A method of treating a disease or condition mediated by propionyl-CoA carboxylase, comprising administering to a subject a propionyl-CoA carboxylase produced using the synthetic polynucleotide of claim 1.

49. The method of claim 47, wherein:
(a) the disease or condition is propionic acidemia (PA);
(b) the polynucleotide is inserted into a cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system) and meganuclease re-engineered homing endonucleases on a cell from the subject; and administering the cell to the subject; or
(c) the composition is administered subcutaneously, intramuscularly, intradermally, intraperitoneally, or intravenously.

50. (canceled)

51. A method of treating a disease or condition mediated by propionyl-CoA carboxylase, comprising administering to a subject a propionyl-CoA carboxylase produced using the rAAV of claim 7, optionally wherein the composition is administered through the route consisting of subcutaneously, intramuscularly, intradermally, intraperitoneally, and intravenously.

52-53. (canceled)

54. The method of claim 47, wherein:
(I) the rAAV is administered at a dose of about 1×10¹¹ to about 1×10¹⁴ genome copies (GC)/kg; or
(II) administering the rAAV comprises administration of a
(a.) single dose of rAAV, or
(b.) multiple doses of rAAV.

55. (canceled)
